(19) United States
(12) Patent Application Publication
(10) Pub. No.: US 2026/0007434 A1
(43) Pub. Date: Jan. 8, 2026

(54) LOCKABLE PIVOTAL BONE ANCHOR ASSEMBLY HAVING PRE-LOCK FIXED FORCED DOWNWARD DISPLACEMENT OF INSERT BY TOOLING

(71) Applicant: Roger P. Jackson, Prairie Village, KS (US)

(72) Inventors: Roger P. Jackson, Prairie Village, KS (US); James L. Surber, Kansas City, KS (US)

(21) Appl. No.: 19/326,081

(22) Filed: Sep. 11, 2025

Related U.S. Application Data
(63) Continuation-in-part of application No. 19/082,087, filed on Mar. 17, 2025.
Application No. 19/082,087 is a continuation of application No. 18/489,811, filed on Oct. 18, 2023, now Pat. No. 12,251,138.
Application No. 18/489,811 is a continuation of application No. 17/571,208, filed on Jan. 7, 2022, now Pat. No. 11,793,553.
Application No. 17/571,208 is a continuation of application No. 16/259,905, filed on Jan. 28, 2019, now Pat. No. 11,219,471.
Application No. 16/259,905 is a continuation of application No. 15/521,163, filed on Apr. 21, 2017, now Pat. No. 10,188,432.
(60) Provisional application No. 62/066,813, filed on Oct. 21, 2014.
(60) Provisional application No. 62/066,806, filed on Oct. 21, 2014.
(60) Provisional application No. 62/078,173, filed on Nov. 11, 2014.
(60) Provisional application No. 62/078,154, filed on Nov. 11, 2014.
(60) Provisional application No. 62/137,713, filed on Mar. 24, 2015.
(60) Provisional application No. 62/137,707, filed on Mar. 24, 2015.
(60) Provisional application No. 62/194,955, filed on Jul. 21, 2015.
(60) Provisional application No. 62/200,501, filed on Aug. 3, 2015.
(60) Provisional application No. 62/200,491, filed on Aug. 3, 2015.
(60) Provisional application No. 62/212,253, filed on Aug. 31, 2015.

(51) Int. Cl. A61B 17/70; A61B 17/86; A61B 90/00

(52) U.S. Cl. CPC A61B 17/7037; A61B 17/702; A61B 17/7035; A61B 17/7038; A61B 17/8605; A61B 17/863; A61B 17/864; A61B 2090/037

(57) ABSTRACT

A pivotal bone anchor assembly includes a receiver having a central opening with opposed arcuate attachment grooves and opposed arcuate recessed surfaces between the opposed arcuate attachment grooves and a lower support surface. The assembly also includes an insert having opposite outwardly-facing side surfaces with arcuate projecting structures positionable in the opposed arcuate attachment grooves in a first position, with upper and lower overlapping interference abutment surfaces inhibiting substantial axial movement within the central opening in either direction. The assembly further includes a bone attachment structure having a capture head pivotally positionable in the lower portion of the receiver in an unlocked configuration, after which the insert is configured for forced downward displacement to a second position in which the arcuate projecting structures are below the lower overlapping interference abutment surfaces so as to inhibit upward displacement of the insert back to the first position. The bone attachment structure remains pivotal with respect to the receiver after the insert is displaced into the second position and until the assembly is locked with a closure top.

### CROSS-REFERENCE TO RELATED APPLICATIONS

This application is a continuation-in-part of U.S. application Ser. No. 19/082,087, filed on Mar. 17, 2025, which is a continuation of U.S. application Ser. No. 18/489,811, filed Oct. 18, 2023, now U.S. Pat. No. 12,251,138, which is a continuation of U.S. application Ser. No. 17/571,208, filed Jan. 7, 2022, now U.S. Pat. No. 11,793,553, which is a continuation of U.S. application Ser. No. 16/259,905, filed Jan. 28, 2019, now U.S. Pat. No. 11,219,471, which is a continuation of U.S. application Ser. No. 15/521,163, filed Apr. 21, 2017, now U.S. Pat. No. 10,188,432, which is a national stage entry of PCT Application No. PCT/US2015/56706, filed Oct. 21, 2015, each of which is incorporated by reference in its entirely herein and for all purposes.

The PCT Application No. PCT/US2015/56706 also claims priority to and the benefit of U.S. Provisional Application No. 62/212,253, filed Aug. 31, 2015, U.S. Provisional Application No. 62/200,501, filed Aug. 3, 2015, U.S. Provisional Application No. 62/200,491, filed Aug. 3, 2015, U.S. Provisional Application No. 62/194,955, filed Jul. 21, 2015, U.S. Provisional Application No. 62/137,713, filed Mar. 24, 2015, U.S. Provisional Application No. 62/137,707, filed Mar. 24, 2015, U.S. Provisional Application No. 62/078,173, filed Nov. 11, 2014, U.S. Provisional Application No. 62/078,154, filed Nov. 11, 2014, U.S. Provisional Application No. 62/066,813, filed Oct. 21, 2014, and U.S. Provisional Application No. 62/066,806, filed Oct. 21, 2014, each of which is incorporated by reference in its entirety herein and for all purposes.

The following applications are related to the present application and hereby incorporated by reference in their entireties into the present application: U.S. application Ser. No. 14/181,998, filed on Feb. 17, 2014, titled; U.S. Provisional Application No. 61/456,163, filed Nov. 2, 2010, U.S. Provisional Application No. 62/007,616, filed Jun. 4, 2014, U.S. Provisional Application No. 61/336,911, filed Jan. 28, 2010, U.S. application Ser. No. 13/317,969, filed Nov. 1, 2011, U.S. application Ser. No. 14/164,882, filed on Jan. 27, 2014, U.S. patent application Ser. No. 11/140,343, filed on May 27, 2005, U.S. application Ser. No. 12/148,465, filed on Apr. 18, 2008, U.S. application Ser. No. 13/573,516, filed Sep. 19, 2012, U.S. application Ser. No. 13/694,954, filed Jan. 22, 2013, and U.S. application Ser. No. 14/061,393, filed on October 2013.

### TECHNICAL FIELD

The present disclosure generally relates to pivotal bone anchor assemblies for use in bone surgery, particularly spinal surgery, and associated their methods of manufacture and use.

### BACKGROUND

Polyaxial bone screws and related anchors of various types have been used for supporting rods and other elongate members in spinal surgery.

Bone screws are utilized in many types of spinal surgery in order to secure various implants to vertebrae along the spinal column for the purpose of stabilizing and/or adjusting spinal alignment. Although both closed-ended and open-ended bone screws are known, open-ended screws are particularly well suited for connections to rods and connector arms, because such rods or connecting members do not need to be passed through a closed bore, but rather can be laid or urged into an open channel within a receiver or head of such a screw.

Typical open-ended bone screws include a threaded shank with a pair of parallel projecting branches or arms which form a yoke with a U-shaped slot or channel to receive a rod. Hooks and other types of connectors, as are used in spinal fixation techniques, may also include open ends for receiving rods or portions of other structure.

Open-ended bone screws or anchors of this type may have a fixed head or a separate head or a receiver relative to a shank thereof. In the fixed bone screws, the rod receiver head cannot be moved relative to the shank and the rod must be favorably positioned in order for itself to be placed within the receiver head. This is sometimes very difficult or impossible to do. Therefore, multiaxial or polyaxial, uniplanar or monoplanar, and biplanar bone screws or anchors are commonly preferred. Open-ended polyaxial bone anchors or screws typically allow for a loose or floppy rotation of the head or receiver about the shank until a desired rotational position of the head is achieved by fixing such position relative to the shank. during a final stage of a medical procedure when a rod or other longitudinal connecting member is inserted into the head or receiver, followed by a locking screw or other closure.

Some of these open-ended bone screws utilize a lower pressure insert positioned within a separate receiver to transfer locking forces from a rod, longitudinal member, or other structure above the insert to a separate shank head below the insert, so as to lock the shank in a fixed angular configuration with respect to a receiver, a receiver assembly or receiver subassembly. Again, the receiver assemblies can be configured as a polyaxial, uniplanar, or biplanar receiver assemblies. There is a need to have different types of bone attachment structures, such as hooks, and screw shanks, that share the same type of upper portion capture structures, so as to be able to connect with these different types of receiver assemblies for cost efficiency and logistical reasons. Shank heads have been designed with flat sides, for example, to work with only monoplanar or uniplanar receivers or receiver assemblies, and they have not also been compatible with needed configurations of multiaxial or polyaxial receivers. Therefore, there exists a need to devise receiver assemblies for multiplanar, uniplanar, and biplanar applications.

### SUMMARY

An embodiment according to the invention includes: a pivotal bone screw or anchor apparatus or assembly that includes a receiver subassembly having: a wave spring; a receiver; a non-pivoting retainer; and a compression or pressure insert. The receiver subassembly is configured to work with a multitude of different bone attachment structures having upper portion capture connections or structures. These capture connections include, but are not limited to: an integral spherical head, an integral spherical head with shaved sides usually associated with uniplanar or monoplanar screws, and a spherical head with shaved sides cooperating with different types of pivoting retainer structures, such as, but not limited to: 1) a more narrowed retaining ring or 2) a retaining ring with opposite spherical side structure that creates a more complete spherical form when joined with the shank capture structure. The shank head upper capture portion that cooperates with a retaining structure, and that pivots with and without shaved or flat sides, has at least these configurations for an interface surface: cylindrical, conical, frusto-conical, or curvate.

A preferred embodiment of the invention includes a bone anchor assembly according to the invention and includes: a shank head having an upper capture portion configured as a partially spherically shaped structure with opposite parallel flat planar surfaces; a receiver having a centrally aligned lower aperture opening onto a bottom surface thereof, the lower aperture being capable of receiving and capturing the shank head; a non-pivoting retainer, the non-pivoting retainer being located in a receiver first locking chamber and being expandable in a receiver second expansion chamber, such that the non-pivoting retainer is capable of expanding about the shank head, so as to capture the shank; a compressible wave spring being positioned within the receiver second chamber, and located above the non-pivoting retainer; a compression insert engageable with the shank upper capture portion and located there above, and wherein the wave spring or other similar structure stabilizes and controls the position and alignment of the non-pivoting retainer structure, both vertically and rotationally within the receiver.

Further embodiments include a pivoting retainer on the shank head. The pivoting retainer is envisioned to be ring shaped and is expanded about the shank head upper capture portion and mated against an interface surface, the interface surface being sized and shaped to mate with an internal surface of the pivoting retainer. In some embodiments, the pivotal retainer may include super structures or vertical extensions on opposite sides thereof, the super structures being substantially planar on a respective internal surface thereof and partially spherical on respective outer surfaces thereof such that the shank and pivoting retainer in combination create a substantially spherical outer surface.

In several of the illustrated embodiments the interface surface of the shank is cylindrically shaped, conically shaped, curvate shaped, or frusto-conically shaped, such that the interface surface has a smaller diameter near a top of the shank.

In the illustrated embodiments the wave spring is in spaced relation with respect to the shank head, but it is foreseen that the wave spring and shank may engage in some embodiments.

It is envisioned that at least one of the receiver interior, the pivotal retainer, the wave spring, the non-pivotal lower retainer, the interface surface and the outer surface of the shank includes a surface treatment, such as knurled, scored, roughened, grit blasted, and textured.

It is foreseen that the bone anchor assembly of the present invention may include bone screws, bone hooks, and other bone attachment structures, such as clamps and ligaments, in both monoplanar and multiplanar configurations.

It is foreseen that the lower compression insert has at least one surface engaging the receiver to block axial rotation therebetween, and wherein the insert can provide either a friction fit or floppy fit when the shank is in an unlocked orientation with respect to the receiver.

In some of the illustrated embodiments, the insert can be bottom loaded into the receiver and includes an outer structure that is sized and shaped to mate with a receiver aperture or groove located on a receiver internal surface, such that when the insert outer structure is mated with the receiver aperture, the insert is captured with respect to the receiver, and prevented from further moving up and down within the receiver until a force is applied to move the insert down.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an exploded view of an embodiment of a bone screw assembly according to the present invention including a shank, a receiver, upper pivoting and lower non-pivoting retainers, a wave spring, a pressure insert and a closure, shown in conjunction with a rod.

FIG. 2A is a cross-sectional view of the bone screw taken along line 2A-2A of FIG. 3.

FIG. 2B is an enlarged fragmentary side view of the bone screw shank of FIG. 1.

FIG. 3 is a top plan view of the bone screw shank.

FIG. 4 is a perspective view of an upper pivoting retainer of the assembly with a portion shown in phantom.

FIG. 5 is a fragmentary perspective view of the upper pivoting retainer.

FIG. 6 is a fragmentary perspective view of the bone screw and the upper retainer, with the upper retainer being expanded about the bone screw upper portion.

FIG. 7 is an enlarged perspective view of the receiver of the assembly.

FIG. 8 is a bottom plan view of the receiver.

FIG. 9 is a top plan view of the receiver.

FIG. 10 is a cross-sectional view of the receiver, taken along line 10-10 of FIG. 9.

FIG. 11A is a perspective view of a non-pivoting retainer in a first embodiment of the assembly.

FIG. 11B is a perspective view of a second embodiment of the lower non-pivoting retainer.

FIG. 11C is a perspective view of a third embodiment of the non-pivoting retainer.

FIG. 12 is a transverse cross-sectional view of the retainer.

FIG. 13A is a perspective view of a first embodiment of a wave spring in the assembly.

FIG. 13B is a perspective view of a second embodiment the wave spring.

FIG. 13C is a perspective view of a third embodiment of the wave spring.

FIG. 14A is a side view of the wave spring shown in a neutral starting state or

configuration.

FIG. 14B is a side view of the wave spring shown in a compressed state or configuration.

FIG. 15 is a perspective view of a compression insert of the assembly.

FIG. 16 is a top plan view of the insert.

FIG. 17 is a bottom plan view of the insert.

FIG. 18 is a side elevation view of the insert.

FIG. 19 is a side elevation view of the receiver and insert with portions of the receiver cut away to show cooperation of the parts at a stage whereat the insert is positioned in the receiver.

FIG. 20 is a perspective of the receiver and insert with portions of the receiver cut away to show cooperation of the parts at a stage whereas the insert is in a higher position relative to the receiver than FIG. 19 and the insert corners are guided by the guides of the receiver and the lower collet portion is being compressed.

FIG. 21 is a side elevation view of the receiver and insert with portions of the retainer broken away to illustrate mating of the insert and retainer.

FIG. 22 is a perspective view of the receiver, insert, and wave spring with portions of the receiver cut away to show the cooperation of the parts at a stage when the wave spring is being loaded into the receiver lower opening.

FIG. 23 is a side elevation view of the receiver, insert, non-pivoting retainer and wave spring with portions of the receiver cut away to show the cooperation of the parts at a stage when the non-pivoting retainer is being loaded into the receiver lower opening.

FIG. 24 is a side elevation view of the receiver, insert, non-pivoting retainer, shank, and wave spring with portions of the receiver cut away to show the cooperation of the parts at a stage when the shank and upper pivoting retainer in combination are loaded into the receiver lower opening.

FIG. 25 is a view similar to FIG. 24 showing a second stage of the positioning of the lower non-pivoting retainer in the receiver, wherein the lower non-pivoting retainer is expanded about the shank upper portion and the upper pivoting retainer, the lower non-pivoting retainer now being positioned in the receiver expansion chamber.

FIG. 26 is a fragmentary cross-sectional view of the receiver as in FIG. 24 showing a third stage of the positioning of the lower non-pivoting retainer as it returns to the second chamber of the receiver in response to downward pressure from the wave spring. The insert is frictionally engaging the shank head upper capture portion and upper pivotal retainer in the insert lower collet portion.

FIG. 27 is a greatly enlarged fragmented cross-sectional view of the receiver as in FIG. 24, showing a fourth stage of the positioning of the insert in the receiver with the shank head and upper pivoting retainer having moved downward to now engage the non-pivoting retainer, wherein the insert collet bottom surface engages and compresses the wave spring to further hold down the lower non-pivoting retainer.

FIG. 28A is a magnification of a portion of FIG. 27, designated as 28AB, in which the insert protrusion structure is shown in a friction fit embodiment against the receiver internal surface.

FIG. 28B is a magnification of a portion of FIG. 27, designated as 28AB, in which the insert receiver attachment structure is shown in a floppy fit embodiment.

FIG. 28C is a magnification of a portion of FIG. 27, designated as 28C, in which the insert lower collet portion has an inner cylindrical surface at its bottom opening, so as to not need to expand around the pivoting retainer when it is inserted therein.

FIG. 29 is a side elevation view of the bone anchor assembly of FIG. 1 with portions of a vertebra broken away to show the detail thereof, shown with a driving tool in a stage of implantation of the shank into the vertebra.

FIG. 30 is a view similar to FIG. 29, showing a receiver in phantom being inserted onto the upper portion of a previously implanted shank head.

FIG. 31 is a side elevation view of the bone anchor assembly of FIG. 1 with portions of a vertebra broken away to show the detail thereof, showing an early stage of insertion with the shank pre-assembled to the receiver subassembly. The compression insert, wave spring, and upper pivoting and lower non-pivoting retainers, are shown with portions broken away and a driving tool shown in a stage of implantation of the bone screw assembly into a vertebra.

FIG. 32 is a cross section of the receiver as in FIG. 24 showing a fifth stage of the positioning of the compression insert in the receiver, with a spinal fixation rod of a first diameter and related closure added, with the closure applying pressure to the rod.

FIG. 33 is a view similar to FIG. 32 showing a fifth stage of the positioning of the insert in the receiver with a spinal fixation rod of a smaller second diameter and the same closure added, again, with the closure applying pressure to the rod.

FIG. 34 is a perspective view of the entire assembly of FIG. 1 shown with the shank at an angle with respect to the receiver.

FIG. 35 is an enlarged and partial side elevation view of the assembly of FIG. 34 with portions broken away to show the detail thereof.

FIG. 36 is a greatly enlarged fragmented perspective view of a second embodiment of the upper pivotal retainer with a portion shown in phantom.

FIG. 37 is a fragmentary perspective view of the second embodiment of the upper retainer of FIG. 35.

FIG. 38 is a perspective view of second embodiment for a bone attachment structure in the form of a hook having an integral spherical head in the shape of a ball.

FIG. 39 is a front side elevation view of the hook of FIG. 38.

FIG. 40 is a side elevation view of a bone anchor assembly employing the hook of FIGS. 38 and 39, with portions broken away to show the detail thereof.

FIG. 41 is a side elevation view of a third embodiment of a shank for use in a bone screw assembly of the present invention.

FIG. 42 is a transverse cross-sectional view of the shank in the third embodiment of FIG. 41.

FIG. 43 is an enlarged fragmentary side elevation view of the third embodiment of the bone anchor assembly, similar to the assembly of FIG. 35, with portions broken away to show the detail thereof.

FIG. 44 is an exploded elevation view of a fourth embodiment of a shank with a third modified embodiment of an upper retainer for use therewith a bone screw assembly of the present invention.

FIG. 45 is a side elevation view of the shank of the fourth embodiment mated with the upper retainer in the third embodiment creating a partially spherical outer surface.

FIG. 46 is a greatly enlarged fragmentary transverse cross-sectional view of the shank in a fourth embodiment mated with the upper retainer in a third embodiment.

FIG. 47 is an enlarged fragmentary cross-sectional view of a bone anchor similar to the bone screw assembly of FIG. 23, with the shank in a fifth embodiment mated with the upper retainer in a fourth embodiment and with portions broken away to show the detail thereof.

FIG. 48 is an enlarged fragmentary cross-sectional view of a bone anchor assembly similar to the assembly of FIG. 23, with the shank in a sixth embodiment mated with the upper retainer in a fifth embodiment and with portions broken away to show the detail thereof.

FIG. 49 is an enlarged fragmentary cross-sectional view of a bone anchor assembly, similar to the bone screw assembly of FIG. 23, with the shank in a seventh embodiment mated with the upper retainer in a sixth embodiment and with portions broken away to show the detail thereof.

The drawings constitute a part of this specification and include exemplary embodiments of the present invention and illustrate various objects and features thereof.

### DETAILED DESCRIPTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

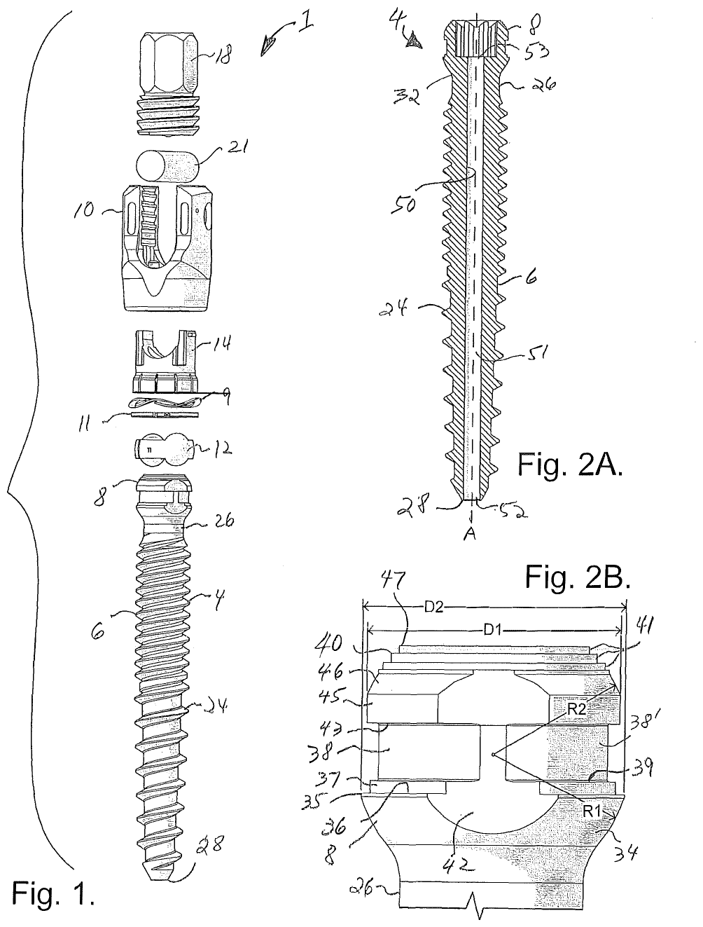

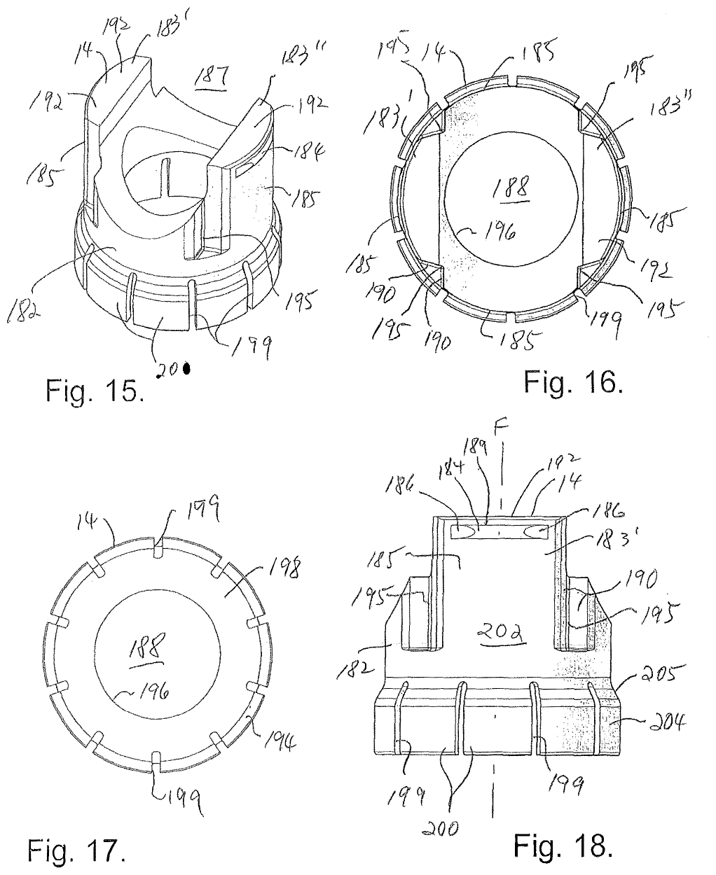

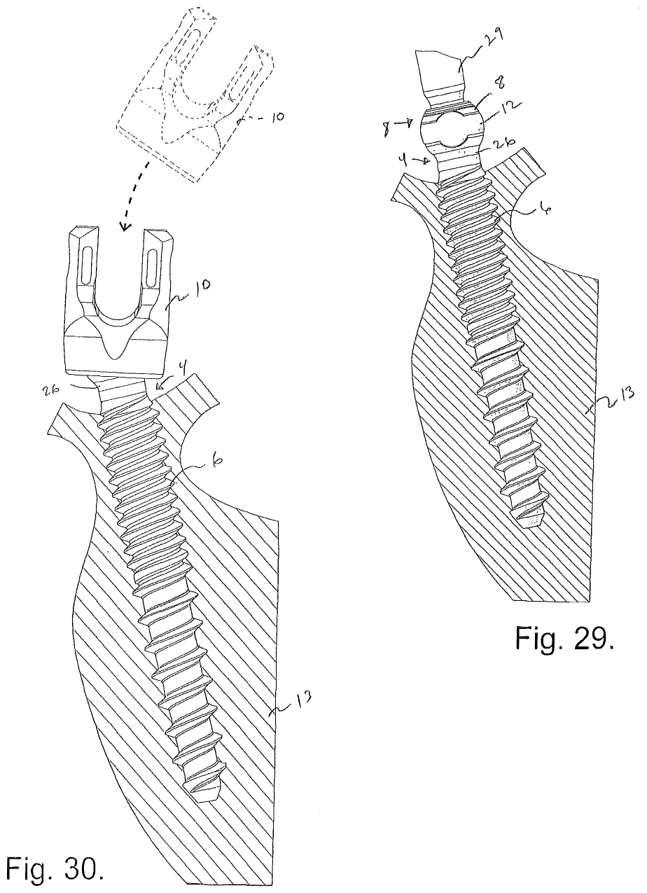

With reference to FIGS. 1-35 the reference number 1 generally represents an embodiment of a multi-planar, multi-axial, or polyaxial bone screw apparatus or assembly according to the present invention. While the illustrated anchor 1 is generally a polyaxial bone screw, it is foreseen that the invention could be utilized with other types of spinal implants that utilize pressure inserts, such as polyaxial bone hooks or clamps, for example. The illustrated assembly 1 includes: a shank 4 that further includes a body 6 integral with an upwardly extending upper portion or capture structure 8; a compressible element or positioner, illustrated as a wave spring 9; a receiver 10; an upper pivoting retainer structure or conversion ring 12; a non-pivoting lower retainer 11; and a compression or pressure insert 14. The receiver 10, the wave spring 9, the non-pivoting retainer 11, and compression insert 14 are initially assembled and may be further assembled with the shank 4 either prior or subsequent to implantation of the shank body 6 into a vertebra 13, as is seen in FIGS. 29-31 and will be described in greater detail below.

FIG. 1 further shows a closure structure 18 of the invention for capturing a longitudinal member, for example, a spinal fixation rod or longitudinal connecting member 21 which in turn engages the compression insert 14 that presses against the shank upper portion or capture portion 8 into fixed frictional contact with the non-pivoting retainer 11, so as to capture, and fix the longitudinal connecting member 21 within the receiver 10 and thus fix the member 21 relative to the vertebra 13. The illustrated rod 21 is hard, stiff, non-clastic and cylindrical, having an outer cylindrical surface 22. It is foreseen that in other embodiments of the invention, the rod 21 may be elastic, deformable and/or of a different cross-sectional geometry. The receiver 10 and the shank 4 cooperate in such a manner that the receiver 10 and the shank 4 can be secured at any of a plurality of angles, articulations or angular alignments relative to one another and within a selected range of angles but not limited to from side to side and from front to rear, to enable flexible or articulated engagement of the receiver 10 with the shank 4 until both are locked or fixed relative to each other near the end of an implantation procedure.

The shank 4, best illustrated in FIGS. 1-3, 6, and 29-31, is elongate, with the shank body 6 having a helically wound bone implantable thread 24 (single or multi start thread forms, which can have different types of thread patterns) extending from near a neck 26 located adjacent to the upper portion or capture structure 8, to a tip 28 of the body 6 and extending radially outwardly therefrom. During use, as best seen in FIGS. 29-31, the body 6 utilizing the thread 24 for gripping and advancement is implanted into the vertebra 13 leading with the tip 28 and driven down into the vertebra with an installation or driving tool 29, so as to be implanted in the vertebra to near the neck 26, as more fully described in the paragraphs below. The shank 4 has an elongate axis of rotation generally identified by the reference letter A in FIG. 2.

The neck 26 extends axially upward from the shank body 6. The neck 26 may be of the same or is typically of a slightly reduced radius as compared to an adjacent upper end or top 32 of the body 6 where the thread 24 terminates. Further extending axially and outwardly from the neck 26 is the shank upper portion 8 that provides a connective or capture apparatus disposed at a distance from the upper end 32 and thus at a distance from the vertebra 13 when the body 6 is implanted in such vertebra.

The shank upper capture portion 8 is configured for a pivotable connection relative to the shank 4 and the upper pivoting retainer 12 with respect to the receiver 10 prior to fixing of the shank 4 in a desired position with respect to the receiver 10. The shank upper portion 8 has an outer bulbous, convex and partially spherical lower surface 34 that extends outwardly and upwardly from the neck 26 and terminates at a lower cylindrical surface 35. The lower cylindrical surface 35 is parallel to axis A and has a diameter or width D2 or radius (not shown) measured from the top most edge from one side to the other, seen in FIG. 2B. It is foreseen that the lower cylindrical surface does not have to be parallel with axis A. The lower cylindrical surface 35 terminates at a substantially planar ledge or shelf surface 36 that is annular and disposed perpendicular to the shank axis A. The spherical lower surface 34 has an outer radius R1 that is the same or substantially similar to an outer radius R3, seen in FIG. 4 of the upper retainer 12, and will be described in greater detail below. The spherical lower surface 34, as well as the upper pivotal retainer 12 outer surface, cooperate to form a ball component 44 of a ball and socket joint formed by the shank 4 and attached upper retainer 12 within the inner surfaces of the receiver 10 defining an inner cavity or space 91, as seen in FIGS. 21-26. Extending upwardly from the ledge 36 is a cylindrical interface surface 38, the surface 38 having a radius or diameter or width that is smaller than the radius R1 of the lower spherical surface 34 and the diameter D2 of the lower cylindrical surface 35.

In the illustrated embodiment of FIG. 2B, there is a secondary step surface 37 that is adjacent to the lower ledge 36 surface. The secondary step surface 37 terminates at a secondary ledge surface 39. It is foreseen that the secondary ledge 39 and step surface 37 can be located adjacent to an upper ledge or shelf 43 rather than the lower ledge 36 as shown. Extending outwardly from the cylindrical interface surface 38 is the annular shelf surface or upper ledge 43 that faces downwardly toward the ledge 36 and is also substantially perpendicular to the axis A. It is envisioned that either the lower ledge 36 or the upper shelf surface 43 may be curved or sloped, as will be discussed in greater detail below. The lower ledge 36, cylindrical interface surface 38 and upper ledge 43 cooperate to capture and fix the resilient open upper retainer 12 to the shank upper portion 8, prohibiting compression of the upper retainer 12 with respect to axis A once the upper retainer 12 is located between the ledges 36 and 43. Extending upwardly from the upper ledge 43 is a cylindrical surface 45 having a width or diameter DI or radius (not shown), measured from the top most edge from one side to the other, seen in FIG. 28. The width or diameter DI or radius (not shown) is illustrated as smaller than the width or diameter D2 or radius (not shown) of the lower cylindrical surface 35, but larger than the radius of the cylindrical interface surface 38. The cylindrical surface 45 width or diameter DI or radius (not shown) is configured for sliding cooperation and ultimate frictional mating with the interior of the upper retainer 12, so as to assist in initiating the expansion of the upper retainer 12.

Extending upwardly from the upper cylindrical surface 45 is an upper partially spherical or domed surface 46. The radius R2 of the upper spherical surface 46 is the same than the radius R1 of the lower spherical surface 34. Located near or adjacent to the surface 45 is an annular top surface 47. It is foreseen that a bevel (not shown) may extend about the spherical surface 46 and may be located between the spherical surface 46 and the annular planar top surface 47.

The spherical surface 46 has the outer radius R2 configured for sliding cooperation and ultimate frictional mating with a concave surface 142 of the compression insert 14 having a substantially similar radius, and also a flat or, in some embodiments, curved surface, as that of the non-pivoting retainer 11, seen in FIGS. 24-27 and will be discussed more fully in the paragraphs below.

The shank top surface 47 is substantially perpendicular to the axis A. The upper spherical surface 46 shown in the present embodiment is substantially smooth with exception of a stepped or graduated upper surface portion 40 located adjacent to the top surface 47 and sized and shaped for cooperation and ultimate frictional engagement with the compression insert 14. In the illustrated embodiment of assembly 1, the surface portion 40 includes at least three graduated cylindrical surfaces 41 disposed substantially parallel to the axis A and adjacent perpendicular step surfaces that are disposed generally perpendicular to the axis A. It is foreseen that the surface portion 40 may include greater or fewer number of stepped surfaces and that the stepped surfaces be further structure rather than carved into the shank spherical surface 34. It is also foreseen that similar stepped surfaces could be carved in and winding helically about on the spherical surface 34 near the neck 26 as envisioned in U.S. patent application Ser. No. 14/164,882, which is incorporated by reference herein. It is foreseen that the surface portion 40 and also the rest of the spherical surface 46 may additionally or alternatively include a roughened or textured surface or surface finish, or may be scored, knurled, grit blasted, or the like, for enhancing frictional engagement with the non-pivoting retainer 11 and/or the compression insert 14.

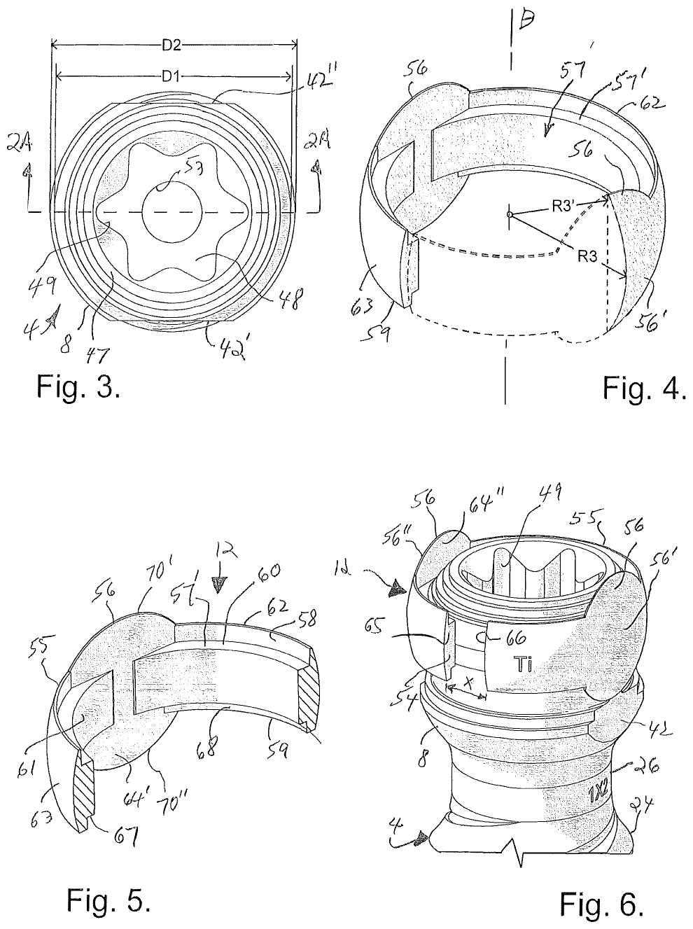

Formed in a shank upper portion 8 of the shank head 4 are opposite or opposed parallel flat planar surfaces 42′ and 42″ that extend downwardly in the direction of axis A and separate the shelf surface 43 into two opposed and spaced apart surfaces. In the illustrated embodiment of the shank in FIG. 3., the flat planar surfaces 42′ and 42″, hereafter referred to as planar surfaces 42 (as seen in FIG. 2B) are machined or molded, such that the width between the flat planar surfaces 42 is less than or substantially equal to the width or radius or diameter (not shown) of the interface surface 38, or now separated surfaces 38 and 38′. The opposite and parallel flat planar surfaces 42 create a disconnect, such that the upper shelf and ledge surface 43, the upper spherical surface 46, the upper cylindrical surface 45, the interface surface 38, the secondary step surface 37, the lower spherical surface 34, the lower cylindrical surface 35, and the lower ledge surface 36 are all discontinuous. Each of the upper ledge surface 43 and the lower ledge surface 36 stop at each of the flat planar surfaces 42 and continue after along the circular path created by the substantially spherical surfaces. It is foreseen that the flat planar surfaces 42 may also include a lower key extension, as seen in U.S. patent application Ser. No. 13/573,516, the entirety of which is incorporated by reference herein. The upper pivoting retainer 12 can be top, bottom, or side loaded onto the shank head. Once the shank head 8 passes through the upper retainer 12, for example, by top loading, the planar side surfaces 42 mate with the upper retainer 12 creating a spherical ball shape structure 44, as will be further described below. In this way, the pivoting retainer 12 is prevented from rotating along the axis A with respect to the shank.

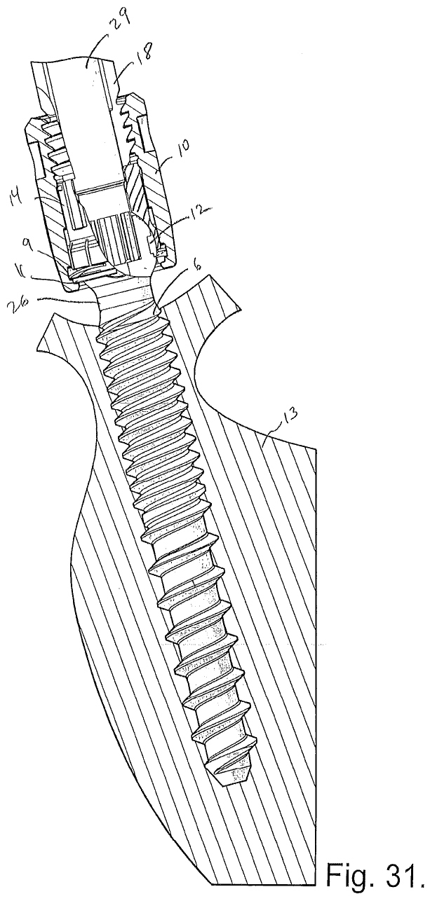

A counter sunk substantially planar base or seating surface 48 partially defines an internal drive feature or imprint or structure 49. As best seen in FIG. 3, the illustrated internal drive feature 49 is an aperture formed in the top surface 47 extending downwardly from the top surface 47 and has a hexagonal or hex shape designed to receive the hex tool 29 of an Allen wrench type, into the aperture for rotating and driving the bone screw shank 4. It is foreseen that such an internal tool engagement structure may take a variety of tool-engaging forms and may include one or more apertures of various shapes, such as a pair of spaced apart apertures or a multi-lobular or star-shaped aperture, such as those sold under the trademark TORX, or the like, having a non-round shape for positive drive engagement by a complementary shaped drive tool. It is foreseen that the drive tool structure may be made of a somewhat softer metal compared to that of the head. The seat or base 48 of the drive feature 49 is disposed perpendicular to the axis A with the drive feature 49 otherwise being coaxial with the axis A. In operation, the driving tool 29 is received in the internal drive feature 49, being seated at the base 48 and engaging the six faces of the drive feature 49 for both driving and rotating the shank body 6 into the vertebra 13, either before the shank 4 is attached to the receiver 10 as shown in FIG. 29 or after the shank 4 is attached to the receiver 10 as shown in FIG. 26, with the shank body 6 being driven into the vertebra 13 with the driving tool extending into the receiver 10, as shown in FIG. 31.

The shank 4 shown in the drawings is cannulated, having a small central bore 50 extending an entire length of the shank 4 along the axis A. It is foreseen that the central bore may not have to extend in a parallel direction with A, that the bore may not extend the entire length of the shank 4. The bore 50 is defined by an inner cylindrical wall or surface 51 of the shank 4 and has a circular opening 52 at the shank tip or end 28 and an upper opening 53 communicating with the external drive 49 at the surface 48. The bore 50 is coaxial with the threaded body 6 and the upper portion 8. The illustrated bore 50 provides a passage through the shank 4 interior for a length of wire (not shown) inserted into the vertebra 13 prior to the insertion of the shank body 6, the wire providing a guide for precise insertion of the shank body 6 into the vertebra 13.

It is foreseen that the shank can be expandable and/or fenestrated, and again, have different thread patterns extending along its length. It is foreseen that the length of the shank may be shortened or lengthened further.

To provide a biologically active interface with the bone or vertebra 13, the threaded shank body 6 may be coated, perforated, made porous or otherwise treated. The treatment may include, but is not limited to, a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄))₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

With particular reference to FIGS. 4-6, the upper pivotal retainer 12 operates to assist in capturing the shank upper portion 8 within the receiver 10. The upper pivotal retainer 12 has a central axis B that operationally aligns with axis A associated with the shank 4 when assembled thereon, as best seen in FIG. 4. The upper pivotal retainer 12 is ring shaped, having a central bore 57, and made from a resilient material, such as a stainless steel, titanium alloy, cobalt chrome, or the like, as well as polymers, so that a retainer body 55 may be resiliently expanded.

The upper pivotal retainer 12 includes a substantially cylindrical continuous body 55 except for a slot or slit 54 and a pair of opposed, opposite, circular semi-spherically shaped surfaces 56′ and 56″. In this pivoting retainer embodiment, surfaces 56′ and 56″ extend upwardly and downwardly from the body 55 of the upper retainer 12 along a radius R3′ measured from the center of the bore 57 that matches with and is equivalent to and form from the radius R3 of the outer surface 63 of the upper retainer 12. It is foreseen that the radius R3 may be slightly smaller or slightly larger than R1 and R2 or some combination thereof, but generally R1, R2, and R3 are identical.

It is foreseen that the spherical surface 63 may additionally or alternatively include a roughened or textured surface or surface finish, or may be scored, knurled, grit blasted, or the like, for enhancing frictional engagement with the components within the receiver 10. It is noted that the surfaces 63 need not be spherical and may be planar, tapered, or faceted or include other surface geometries, such as conical.

The upper retainer body 55, having only the very narrow slit 54 to be used for expansion purposes only when the shank upper capture portion 8, is loaded in combination with the shank upper capture portion 8 through the receiver lower opening 136, as best seen in FIG. 24. The bore 57 surfaces are sized and shaped such that the upper retainer body 55 cannot compress further when mated with the shank upper capture portion 8. The through slit 54 of the resilient upper retainer 12 is defined by first and second end surfaces, 65 and 66, respectively, disposed in spaced relation to one another or they may also be touching when the retainer is in a neutral, natural, or nominal starting state or position. Both end surfaces 65 and 66 are disposed substantially perpendicular to a bottom surface 59 and a top surface 62. It is foreseen that the slit 54 may be at an angle or curved. Referring to FIGS. 4 and 6, a width X between the surfaces 65 and 66 is very narrow in the nominal state to provide stability and more surface contact area for the upper retainer 12 during operation. The upper retainer 12 and shank upper capture portion 8 in combination are bottom loaded through the receiver lower opening 136, such that the upper retainer 12 is loaded in a neutral state, such that it does not need and, in fact, cannot be further compressed to fit within a receiver lower opening 136.

Referring now to FIG. 5, the upper retainer 12 hollow through bore 57 passes entirely through the upper retainer 12. Surfaces that define the channel or bore 57 include: an upper cylindrical surface 58 adjacent to the retainer top surface 62, a discontinuous shelf surface 60 adjacent to the upper cylindrical surface 58, a discontinuous cylindrical surface 61 adjacent to the shelf surface 60, a discontinuous sloped or ramped surface 67 adjacent to the discontinuous cylindrical surface 61, opposed inner planar surfaces 64′ and 64″ and communicating with the cylindrical surface 58 and a third cylindrical surface 68. The bore 57 is sized and shaped to closely fit about and snap onto the shank interface surface 38 during assembly. The upper cylindrical surface 58 is continuous about the bore 57 of the upper retainer 12, but for, the inner planar surfaces 64′ and 64″. The inner planar surfaces 64′ and 64″ are the interior surfaces of the semi-spherically shaped surface 56′ and 56″ have a circular portion 70′ adjacent to the top surface 62 communicating with the cylindrical surface 58 and a circular portion 70″ adjacent to the bottom surface 59 communicating with third cylindrical surface 68. The inner planar surfaces 64′ and 64″ run parallel with axis B, which are adjacent and interrupt the bore surfaces 58, 60, 61, 67, and 68, are seen in FIG. 5. This communication of surfaces creates a narrow gap or band 73′ and 73″ where the upper and lower shelf surfaces 36 and 43 terminate at the interface surface 38 of the shank upper capture portion 8 and mate with gap 73′ and 73″ on both opposed sides. The inner surfaces 64′ and 64″ with circular portions circular portion 70′ and 70″ are sized and shaped to mate with and substantially cover the opposed planar surfaces 42 of the shank head upper capture portion 8. When mated the top surface 62 of the pivoting retainer 12 does not extend above the top of the shank top surface 47. The sloped surface 67 does not mate with the secondary ledge 39 and secondary step surface 37, but does engage an edge 71 at the point where the secondary ledge 39 and secondary step surface 37 meet. When the pivoting retainer 12 is mated to shank upper capture portion 8, a ball structure 44 that is substantially spherical is created. Again, the pivoting retainer 12 is stabilized on the shank head upper capture portion 8 with respect to pivotal, rotational, and elevational alignments.

The cylindrical surface 61, shelf surface 60, and the slope surface 67 form a discontinuous annular lug 57′, which is received within a discontinuous annular groove or channel 38′ formed by the interface surface 38 and ledge surfaces 39 and 43 that do not extend entirely around the shank upper capture portion 8, when the upper pivotal retainer 12 is assembled with the shank 4.

It is foreseen that further surfaces such as a lower shelf surface (not shown) and a fourth cylindrical surface (not shown) may be sized and shaped to further step down the spherical shape of the outer surface 63 internally.

It is also foreseen that the semi-spherically shaped surfaces 56′ and 56″ may include projections or notches as needed for tooling to resiliently hold the pivoting retainer 12. It is foreseen that in other embodiments of the invention, fewer or greater number of planar or other surfaces with other geometries may extend between the top surface 62 and the inner surfaces defining the bore 57 of the pivoting retainer 12.

FIG. 6 illustrates how the gap X functions only in expansion to allow the upper retainer 12 to expand about the shank upper portion 8 and the upper retainer 12 and returns to the natural state when fully mated. This results in a stronger retainer that provides more surface contact with the shank upper portion 8 upon locking, resulting in a sturdier connection with less likelihood of failure than a retainer ring having a greater gap or one that is compressible on the shank. Furthermore, because the retainer body 55 is only expanded and not further compressed, the upper retainer 12 does not undergo the mechanical stress that typically is placed on spring ring type retainers that are both compressed and expanded during insertion of the shank upper capture portion 8.

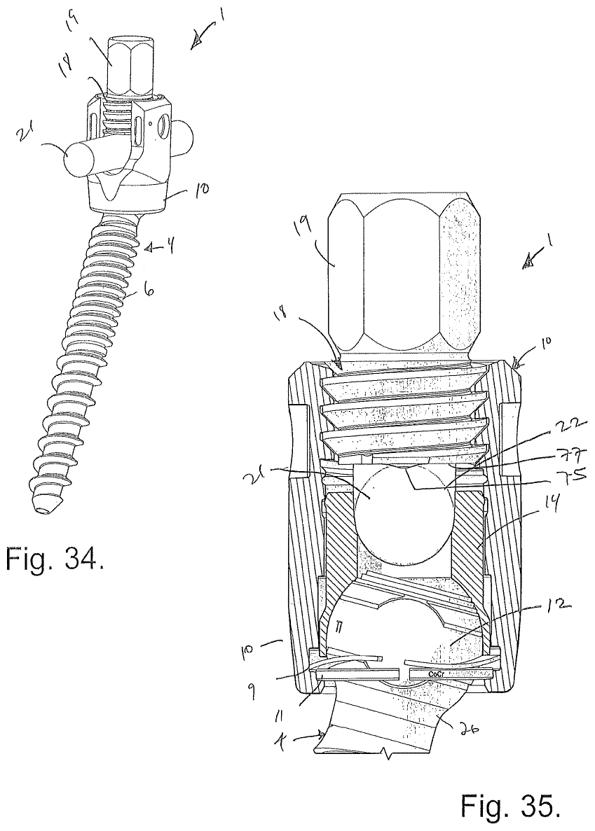

With particular reference to FIGS. 1 and 7-10, the receiver 10 has a generally U-shaped appearance with a partially discontinuous substantially cylindrical inner and outer profile. The receiver 10 has a cylindrical axis C that is shown in FIG. 10 that may align with axis A of the shank 4, and axis B of the pivoting retainer 12, such orientation being desirable, but not required during assembly of the receiver 10 with the shank 4 and pivoting retainer 12. After the receiver 10 is pivotally attached to the shank 4 at a desired predisposed plane or axis, either before or after the shank 4 is implanted in a vertebra 13, the axes B and C are typically disposed at an obtuse angle with respect to the axis A, as shown, for example, in FIGS. 34-35.

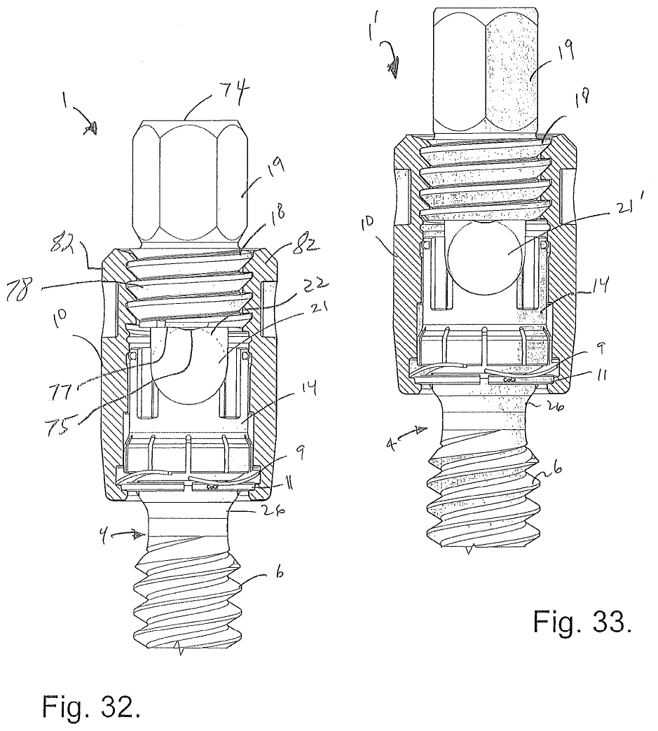

The receiver 10 includes a substantially cylindrical base 80 integral with a pair of opposed upstanding arms 82′ and 82″ forming a cradle and defining a channel 84 between the arms 82′ and 82″ with an upper opening, generally 86, and a U-shaped lower seat 88, the channel 84 having a width for operably snugly receiving the rod 21 between the arms 82′ and 82″, best seen in FIGS. 32-33. Each of the arms 82′ and 82″ has an interior surface, generally 90, that includes various inner cylindrical profiles, an upper of which is a partial helically wound guide and advancement structure 92 located adjacent to top surfaces 93′ and 93″ of each of the arms 82′ and 82″. It is foreseen that the receiver may further include extensions (not shown) attached to the arms 82′ and 82″ having break off junctures to the arms. The breakoff extensions can also have internal threads.

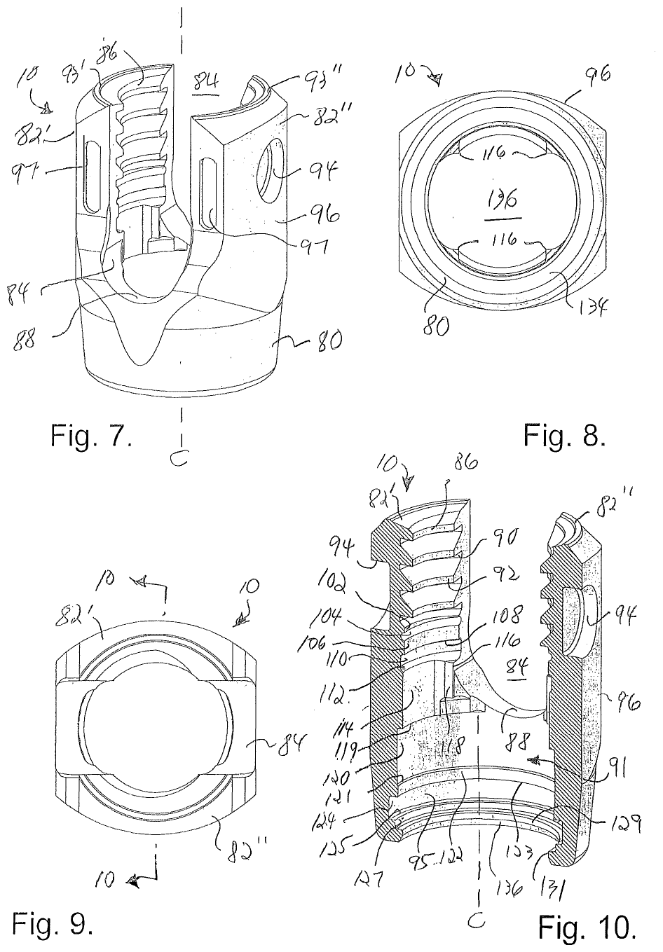

In the illustrated embodiment seen in FIGS. 7-10, the guide and advancement structure 92 is a partial helically wound interlocking reverse angle form configured to mate under rotation with a similar structure on the closure structure 18 with a breakoff head 19, as described more fully below. However, it is foreseen that the guide and advancement structure 92 could alternatively be a square-shaped thread, a buttress thread, a flange form thread or other thread-like or non-thread-like helically wound and non-helically wound discontinuous advancement structure for operably guiding, under complete or partial rotation, and advancing the closure structure 18 downward between the arms 82′ and 82″, as well as eventual torquing when the closure structure 18 abuts against the rod 21. It is also foreseen that the closure need not have a breakoff head 19 in certain embodiments.

An opposed pair of tool receiving and engaging apertures or indentations 94 are formed on outer surfaces 96 of the illustrated arms 82′ and 82″. Furthermore, two pairs of tool receiving and engaging apertures 97 may be formed in front and rear surfaces 78′ and 78″ of the arms 82′ and 82″. Some or all of the apertures 94 and 97 may be used for holding the receiver 10 during the implantation of the shank body 6 into a vertebra when the shank is pre-assembled with the receiver 10, and during assembly of the bone anchor assembly 1 with the rod 21 and the closure structure 18. It is foreseen that tool receiving grooves or apertures 94 and 97 may be configured in a variety of shapes and sizes and be disposed at other locations on the receiver arms 82′ and 82″, such as near the top of the receiver arms in the form of horizontal radiused grooves.

Referring now to FIG. 10, returning to the interior surface 90 of the receiver arms 82′ and 82″, located just below the guide and advancement structure 92 is a discontinuous cylindrical surface 102. The cylindrical surface 102 has a diameter equal to or slightly less than a root diameter of the guide and advancement structure 92. The channel 84 separates the surface 102 from being continuous about the interior surface 90 of the receiver 10. Moving downwardly, in a direction toward the base 80, adjacent to the cylindrical surface 102 is a discontinuous insert attachment structure or groove or slot or spherical surface 104 that extends outwardly from the axis C and runs perpendicular to the axis C. Adjacent to and located below the insert attachment groove surface 104 is a second discontinuous cylindrical surface 106 having a diameter or width or radius equal to or less than the diameter or width or radius of the surface 102. A discontinuous annular surface 108 that provides an abutment surface or stop for capturing the compression insert 14 in the receiver 10 at a first friction fit position is located below and adjacent to the second cylindrical surface 106. The abutment surface 108 is disposed substantially perpendicular to the axis C. Another cylindrical surface 110 is located below and adjacent to the surface 108. The cylindrical surface 110 is oriented substantially parallel to the axis C and is sized and shaped to capture the compression insert 14 as will be described in greater detail below. The surface 110 surrounds the U-shaped channel seat 68 and is by definition discontinuous. The cylindrical surface 110 has a diameter greater than the diameter of the cylindrical surface 102 and the second cylindrical surface 106. A discontinuous sloped surface 112 is located below and adjacent to the cylindrical surface 110, sloping downwardly and inwardly toward the central axis C. A fourth partially cylindrical surface 114 is located below and adjacent to the surface 112. Located on the fourth cylindrical surface 114 are four spaced and radially inward projecting projections or guides 116, each being located on four corners with a side surface 118 that is parallel with the receiver channel 84. The guides 116 cooperate with the insert 14 as will be further discussed below. An upper shelf surface 120 is located below the fourth partially cylindrical surface 114. The upper shelf surface 119 is disposed substantially perpendicular to the axis B and is a bottom surface to the projecting guides 116. A fifth cylindrical surface 120 is below the upper shelf surface 119. The fifth cylindrical surface 120 is continuous about the interior 90 of the receiver 10 and is at least substantially parallel with axis C. In the illustrated embodiment, the fifth cylindrical surface 119 has a diameter that is greater than the radii of the first cylindrical surface 102, the second cylindrical surface 106, the third cylindrical surface 110, and the fourth cylindrical surface 114 described above. A bevel or sloped surface 121 is below the fifth cylindrical surface 120. The bevel slopes towards axis C and downwardly much like the sloped surface 112 located above it. The bevel 121 is smaller in dimension than that of the sloped surface 112. A sixth cylindrical surface 122 is located below and adjacent to the bevel 121. The sixth cylindrical surface 122 has a diameter that is slightly less than the fifth cylindrical surface 120, as is determined by the length of the bevel.

An upper stop surface 123 is disposed in the base 80 and forms a stop for the resilient non-pivoting retainer 11, prohibiting the non-pivoting retainer 11 (when in an uncompressed configuration) from moving upwardly into a space or cavity 91 defined by the cylindrical surface 110 that holds the compression insert 14. A seventh cylindrical surface 124 is located below and adjacent to the surface 123. The cylindrical surface 123 is oriented substantially parallel to the axis C and is sized and shaped to receive an expanded non-pivoting retainer 11 as will be described in greater detail below. A lower abutment surface 125 is located below the seventh continuous cylindrical surface 124. The lower abutment surface 125 is substantially perpendicular to the axis C and faces upwardly toward the upper stop surface 123. The cylindrical surface 124, the stop surface 123, and the lower abutment surface 125 partially define a circumferential recess or expansion chamber 95 that is sized and shaped to house the wave spring 9 and to receive the non-pivoting retainer 11 as it expands around the shank upper portion 8 as the shank upper capture portion 8 moves upwardly toward the channel 84 during assembly. Additionally, the expansion chamber 95 forms a restriction to prevent the non-pivoting retainer 11 and the wave spring 9 from moving upwardly with the shank portion 8, the stop surface 123 preventing the non-pivoting retainer 11 and the wave spring 9 from passing from the expansion chamber 95 into the cavity 91 whether the non-pivoting retainer 11 is in an expanded position, as shown in FIG. 25, or in a neutral or original operative position, as shown in FIG. 24.

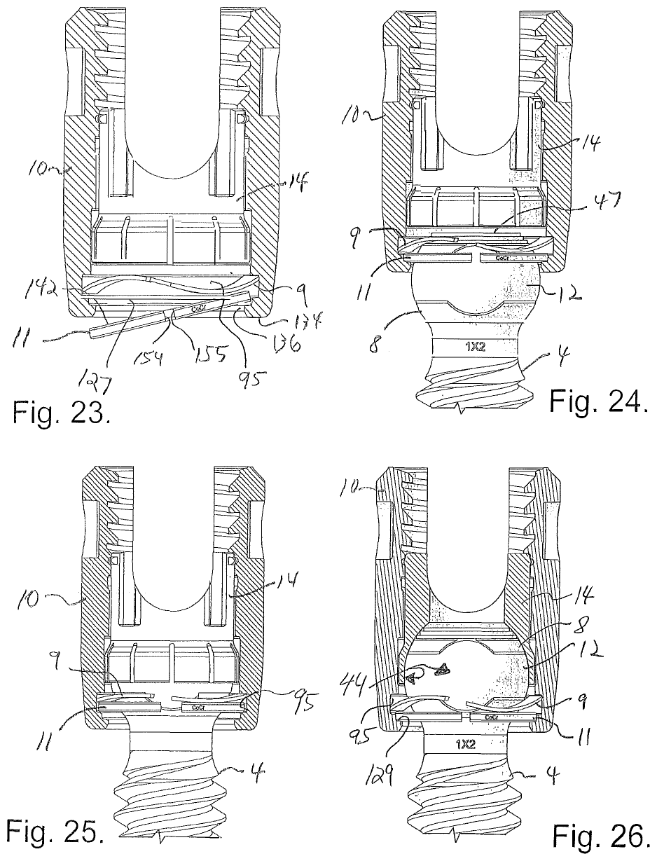

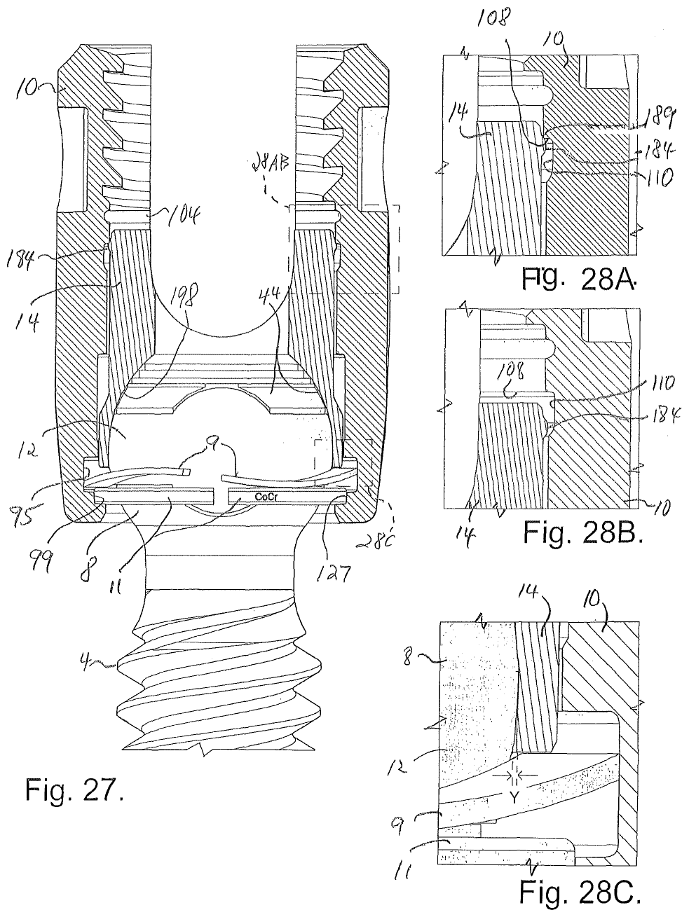

An eighth continuous cylindrical surface 127 is located below the abutment surface 125 of the expansion chamber 95 and is sized and shaped to closely receive the non-pivoting retainer 11 when the retainer is in a neutral or steady or interim position as shown in FIG. 24, for example, or expanded operative position as shown in FIG. 25, for example. Thus, the cylindrical surface 127 has a diameter smaller than the diameter of the seventh cylindrical surface 124 that defines the expansion chamber 95. The surface 127 also has a diameter larger than an outside diameter of the non-pivoting retainer 11 so that the retainer may expand outwardly into contact with the surface 127 when the bone screw shank upper portion 8 presses downwardly during locking of the shank 4 against the non-pivoting retainer 11. The surface 127 is joined or connected to a surface 131 by one or more beveled, curved or conical surfaces. The surfaces 131 allow for sliding and gradual movement and/or contraction of the non-pivoting retainer 11 into the space defined by the surfaces 121, 122, and 123 and ultimate seating of the non-pivoting retainer 11 on a lower annular seating surface 129 located below and adjacent to the cylindrical surface 127. The surfaces 127 and 129 provide a seating or non-expansion chamber for the non-pivoting retainer 11, wherein the retainer slightly expands out to the surface 127 when in a locked position as shown, for example, in FIG. 27. The surface 131 communicates with an exterior base surface 134 of the base 80, defining the lower opening, generally 136, of the receiver 10. The lower opening has a diameter or width D3 or radius (not shown) measured from the upper edge of the curved surface 131. The illustrated surface 131 has a diameter that is substantially the same as the diameter of the surface 110, allowing for slidable uploading of the compression insert 14 while requiring compression or squeezing of the non-pivoting retainer 11 during uploading of the non-pivoting retainer 11 and the wave spring 9 through the lower opening 136 (see FIGS. 22 and 23).

With particular reference to FIGS. 1 and 11A-12, the non-pivoting retainer 11 operates to capture the shank upper portion 8 within the receiver 10. The non-pivoting retainer 11 has a central axis D that is operationally aligned with the axis C associated with the receiver 10, axis B associated with the pivoting retainer 12, and may be aligned with axis A associated with the shank 4. The non-pivoting retainer 11 is made from a resilient material, such as a stainless steel or titanium alloy, cobalt chrome, or the like, or a polymer, so that the non-pivoting retainer 11 may be expanded during various steps of assembly, as will be described in greater detail below. It is foreseen that the non-pivoting retainer 11 may be used for compression as well and held in rotational alignment by the receiver, or a spring ring, a wave spring or other structures, such as additional inserts.

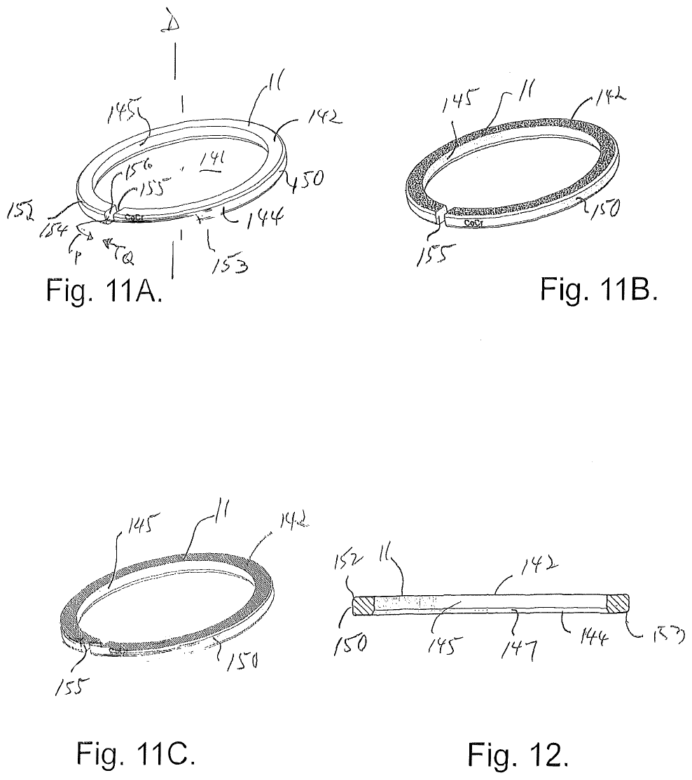

Referring now to FIGS. 11A-C and 12, the illustrated non-pivoting retainer 11 is annular or ring-like in shape and has a central channel or opening or hollow through bore, generally 141, that passes entirely through the non-pivoting retainer 11 from a top surface 142 to a bottom surface 144 thereof. Surfaces that define the channel or bore 141 include a discontinuous inner cylindrical surface 145 adjacent to the top surface 142 and a discontinuous surface or beveled surface 147 adjacent to the inner cylindrical surface 145, both surfaces being coaxial with the axis D when the non-pivoting retainer 11 is in a neutral non-compressed, non-expanded nominal or normal orientation. The non-pivoting retainer 11 further includes an outer cylindrical surface 150 located adjacent to a top outer corner surface 152 which is adjacent to the top surface 142 and a lower outer surface 153 adjacent to the bottom surface 144. The outer surface 150 is oriented parallel to the axis D. It is foreseen that the corners 152 and 153 could be rounded or beveled as needed. It is also foreseen that two or more evenly spaced notches (not shown) may be formed in the cylindrical surface 150 to more evenly distribute stress across the entire non-pivoting retainer 11 during contraction and expansion thereof. In other embodiments of the invention, the notches may be on the inside of the non-pivoting retainer ring 11 or they may be omitted, as shown in FIGS. 11A-C and 12. The resilient non-pivoting retainer 11 further includes first and second end surfaces, 154 and 155 disposed in opposed spaced relation to one another when the retainer is in a neutral non-compressed state. Both end surfaces 154 and 155 are disposed substantially perpendicular to the top surface 122 and the bottom surface 124 and parallel with axis D. The embodiment shown in FIGS. 11A-C and 12 illustrates the surfaces 154 and 155 as substantially parallel; however, it is foreseen that it may be desirable to orient the surfaces obliquely or at a slight angle depending upon the amount of compression desired during loading of the non-pivoting retainer 11 into the receiver 10.

A gap 156 of width X′ between the surfaces 154 and 155 is determined by a desired amount of compressibility of the open non-pivoting retainer ring 11 when loaded into the receiver 10. The gap 156 shown in FIG. 11A provides adequate space between the surfaces 154 and 155 for the non-pivoting retainer 11 to be pinched, with the surfaces 154 and 155 compressed toward one another (as shown by arrows P and Q in FIG. 11A) to a closely spaced or even touching configuration, if necessary, to an extent that the compressed non-pivoting retainer 11 is up loadable or bottom loadable through the receiver opening 136 as shown in FIG. 23. After passing through the opening 136 and along a portion of the lower eighth cylindrical surface 127, the non-pivoting retainer 11 expands or springs back to an original uncompressed, rounded or collar-like configuration of FIGS. 11A-C and 12. It is foreseen that the end portions 152 and 153 can be overlappingly compressed together to get the retainer in the receiver.

It is foreseen that the top surface portion 142 and also the rest of the cylindrical outer surface 150 may additionally or alternatively include a roughened or textured surface or surface finish, or may be scored, knurled (see FIG. 11C), grit blasted (see FIG. 11B), or the like, for enhancing frictional engagement with the eighth cylindrical inner surface 127 of the receiver and a bottom surface 164 of the wave spring 9. The additional surfacing may be necessary to prevent or limit rotational movement of the non-pivotal retainer 11 with respect to the wave spring 9. It is foreseen that the non-pivoting retainer may include further embodiments as seen in U.S. patent application Ser. No. 12/924,802, which is incorporated by reference herein. The retainer may have a super structure or infra-structure extending up and down therefrom.

With particular reference to FIGS. 1 and 13A-14B, the wave spring 9 operates to control, capture and hold the non-pivoting retainer 11 down within the receiver 10 compression or locking chamber. The wave spring 9 has a central axis E that is operationally aligned with axis D associated with the non-pivoting retainer, axis C associated with the receiver 10 (i.e., longitudinal axis of receiver), and the axis B associated with the pivoting retainer 12, and may be aligned with axis A associated with the shank 4. In this way, the positioner or wave spring 9 may maintain coaxial alignment between the axis E of the positioner 9, the axis D of the non-pivoting retainer, and an axis C of the receiver 10. The wave spring may be made from a resilient material, such as a stainless steel, titanium alloy, or the like, as well as polymers, so that the wave spring may be expanded and contracted during various steps of assembly, as will be described in greater detail below.

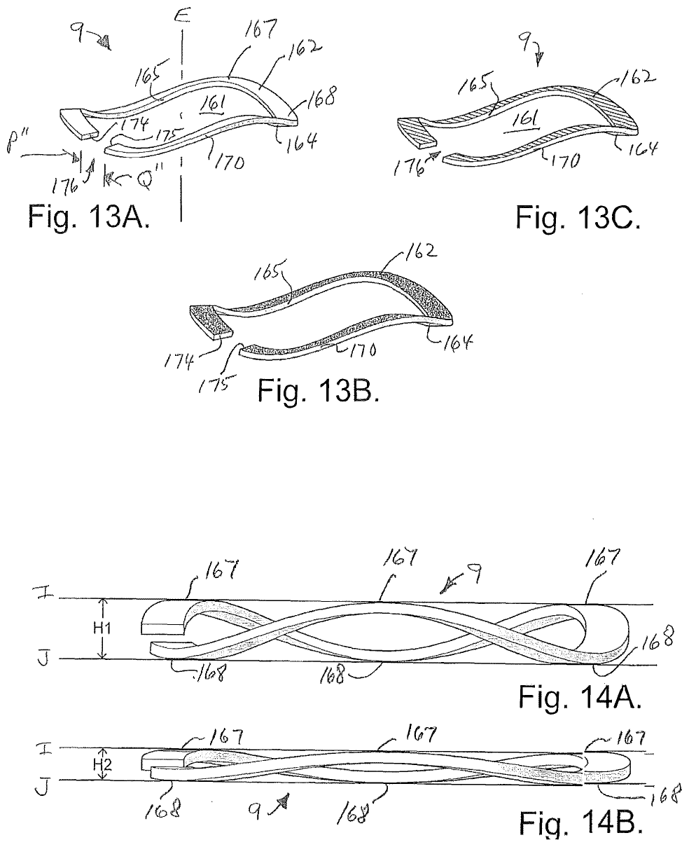

Referring now to FIGS. 13A, the illustrated wave spring 9 is of an annular or ring-like shape and has a central channel, opening, or hollow through bore, generally 161, that passes entirely through the wave spring 9 from a top surface 162 to a bottom surface 164 thereof. Surfaces that define the channel or bore 161 include a discontinuous inner cylindrical surface 165 adjacent to the top surface 162 being coaxial with the axis E when the wave spring 9 is in a neutral non-compressed, non-expanded nominal or normal orientation. The bore 161 is sized and shaped such that inner cylindrical surface 165 does not engage the shank upper portion 8 or the pivoting retainer 12, but could engage the retainer. The non-pivoting retainer 11 further includes an outer cylindrical surface 150 located adjacent to the top surface 162 and the bottom surface 164. The outer surface 150 is oriented parallel to the axis E.

The wave spring 9 further includes first and second end surfaces 174 and 175 disposed in opposed spaced relation to one another when the wave spring 9 is in a neutral non-compressed nominal state or position. Both end surfaces 174 and 175 are disposed substantially perpendicular to the top surface 162 and the bottom surface 164 and parallel with axis E. The embodiment shown in FIGS. 13A-C and 14A-B illustrates the surfaces 174 and 175 as substantially parallel, however, it is foreseen that it may be desirable to orient the surfaces obliquely or at a slight angle depending upon the amount of compression desired during loading of the wave spring into the receiver 10. The surfaces 174 and 175 may also overlap.

A gap 176 having a width X″ separates the surfaces 174 and 175, the width X″ is determined by a desired amount of compressibility of the wave spring when loaded into the receiver 10. The gap 176 shown in FIG. 13A provides adequate space between the surfaces 174 and 175 for the wave spring 9 to be pinched, with the surfaces 174 and 175 compressed toward one another (as shown by arrows P″ and Q″ in FIG. 13A) to a closely spaced or even touching configuration, if necessary, to an extent that the compressed wave spring 9 is up loadable or bottom loadable through the receiver opening 136 as shown in FIG. 22. After passing through the opening 136 and along a portion of the lower seventh cylindrical surface 125, the wave spring expands or springs back to an original uncompressed, rounded or collar-like configuration of FIGS. 13A-14 while within the expansion chamber 95 of the receiver 10.

It is foreseen that the top surface portion 162 and also the rest of the cylindrical outer surface 170 may additionally or alternatively include a roughened or textured surface or surface finish, or may be scored, knurled (see FIG. 13C), grit blasted (see FIG. 13B), or the like, for enhancing frictional engagement with the seventh cylindrical inner surface 125 and the stop surface 123 of the receiver 10 and a top surface 142 of the non-pivoting retainer 11. The additional surfacing may be necessary for the bottom surface of the wave spring to prevent or limit rotational movement of the non-pivotal retainer 11 with respect to the wave spring 9 and also the receiver 10.

The illustrated wave spring or axially resistant structure 9 is a single turn wave with three peaks 167 and valleys 168. Each peak 167 engages the stop surface 123 of the interior 90 of the receiver 10, and each valley 168 engages the abutment surface 125 of the interior 90 of the receiver 10. It is foreseen that the wave spring 9 may be of a different geometry than that of a rectangle cross section. It is foreseen that the wave spring may include further turns such that the valleys 168 of the next turn engage the peaks 167 of the previous turn. It is foreseen that the wave spring 9 may or may not include a ring like structure acting as the top and bottom surfaces of the wave spring. It is foreseen that the wave spring 9 may include further turns, one on top of the other in a nested wave spring (not shown). It is foreseen that more or fewer peaks 167 and valleys 168 could be used in the wave spring 9.

Referring now to FIG. 14A, the illustrated wave spring 9 is shown with the peaks 167 engaging a top plane I and the valleys 168 engaging a bottom plane J, the distance between the planes being height H1.

Referring now to FIG. 14B, the illustrated wave spring 9 of FIG. 14A is shown being compressed such that the distance between the planes I and J is height H2. Height H2 is less than height H1, as shown, with the transition from a neutral state seen in FIG. 14A to a compressed state seen in FIG. 14B. The vertical compression of the illustrated wave spring or vertical resistance structure 9 shifts as the peaks 166 become squished, the overall length of wave spring 9 is transitioned to the width X″, such that end surfaces 174 and 175 are compelled toward each other. In use, the neutral position is where the wave spring 9 is positioned between upper plane I as the stop surface 123 and plane J as the abutment surface 125. In the compressed position, the wave spring 9 is being compressed by the upper plane I as the bottom surface 194 of the insert 14 and the lower plane J as the top surface 142 of the lower non-pivotal retainer 11.

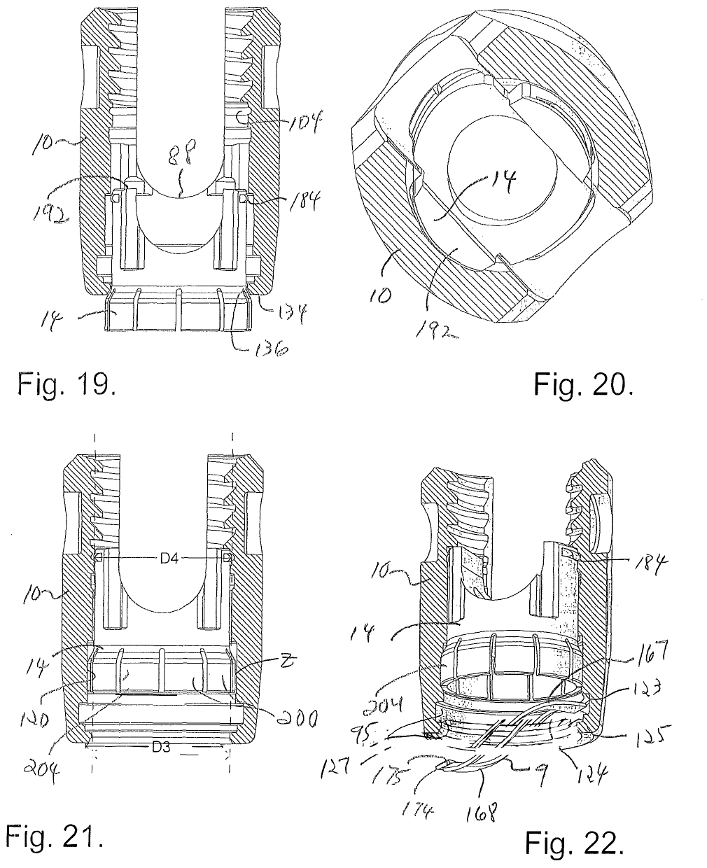

The compression insert 14 is best seen in FIGS. 1 and 15-18. The friction fit compression collet insert 14 that is sized and shaped to be received by and up-loaded into the receiver 10 at the lower opening 136, as seen in FIG. 19. The illustrated compression insert 14 has a central axis F operationally aligned with the central axis C of the receiver 10. In operation, the insert 14 advantageously frictionally engages the bone screw shank upper portion 8, allowing for un-locked but non-floppy placement of the angle of the shank 4 with respect to the receiver 10 during surgery prior to locking of the shank with respect to the receiver near the end of the procedure with a rod or connecting member 21 and a closure 8. The insert 14 is thus preferably made from a resilient material, such as a stainless steel or titanium alloy, so that portions of the insert may be expanded about and then contracted, snapped or popped onto the shank upper portion 8. Furthermore, in operation, the insert 14 is suspended within the receiver 10, being frictionally held in place by the receiver insert attachment grooves 104 and prohibited from moving upward even with the insertion of the wave spring 9, lower insert 11, and the shank upper capture portion 8, as best seen in FIGS. 10, 21, and 22. As will be explained in greater detail below, after initial assembly and during operation of the assembly 1, neither the non-pivoting retainer 11 nor the inner surfaces 90 of the receiver 10 that define the cavity 91 place any compressive force on the insert 14 to hold the shank upper capture portion 8 therein.

The illustrated insert 14 includes a lower body 182 with a pair of spaced upstanding arms 183′ and 183″. The arms 183′ and 183″ have a radially outer surface 185 on each side which are substantially smooth and vertically or axially opposed, but radially spaced from axis F. The outer surface 185 includes receiver attachment projection structures 184 on each arm 183′ and 183″. Each receiver attachment projection structure 184 extends circumferentially about the outer surface and includes a beveled or sloped surface 186 on either or both sides of the projection 184. The projection 184 has a maximum diameter or width or radius (not shown) of D4 measured about the center of the projection 184, as can be seen in FIG. 21. The arms 183′ and 183″ form a central U-shaped channel 187 therebetween, and there is a central axially aligned and centered bore 188. The through bore 188 runs from an annular planar top surface 192 to an annular planar and discontinuous bottom surface 194 thereof. The bore 188 is defined by an inner cylindrical surface 196 that is at least partially defined by the U-shaped channel 187 and a shank gripping surface portion 198 extending between the cylindrical surface 196 and the bottom surface 194. The internal shank gripping surface portion 198 includes a lower cylindrical surface 197 adjacent to the bottom surface 194 and a spherical surface 198′. The cylindrical surface 197 provides a gap Y such that the spherical surface 46 is prevented or limits engagement of the spherical surface 46 and the tabs 200, so as to not further engage and thereby expand the tabs 200, as best seen in FIG. 28C.

It is foreseen that the cylindrical surface 196 and the shank gripping surface 198 may include one or more stepped surfaces (not shown). The compression insert 14 through bore 188 is sized and shaped to receive the driving tool (not shown) there through that engages the shank drive feature 49 when the shank body 6 is driven into bone with the receiver 10 attached or without, see FIGS. 27-29, as will be further discussed below.

It is foreseen that the shank gripping surface portion 198 and also the surface 196 may additionally or alternatively include a roughened or textured surface or surface finish, or may be scored, grit blasted, knurled, or the like (not shown), for enhancing frictional engagement with the shank upper portion 8.

A plurality of slits or slots 199 are formed in the spherical shank gripping surface 198, running through the bottom surface 194 and terminating near or about half way to the cylindrical surface 196. The illustrated embodiment includes ten slots 199. It is foreseen that other embodiments of the invention may include more or fewer slots 199. Each pair of slots 199 forms a distinct resilient, partially spherical finger, tab or panel 200 that extends from the shank gripping portion 198 to the bottom surface 194. In other words, the inner spherical surface 198 is separated into ten surface portions 200, each being partially spherical and sized and shaped to resiliently expand about the upper spherical surface 46 of the shank upper portion 8 and outer spherical surface 63 of the pivoting retainer 12 and then snap on and frictionally grip the surfaces 46 and 63. Preferably, the spherical surface 198 is designed such that the gripping tabs or panels 200 have a neutral or non-expanded radius that is slightly smaller than a radius of the shank spherical surface 46 so that when the tabs or panels 200 are gripping the surfaces 46 and 63, the insert is in a slightly expanded state. The fifth cylindrical surface 120 of the interior 90 of the receiver is dimensioned to allow for this. When the shank 4 is locked into position by a rod or other connecting member 21 being pressed downwardly on the insert U-shaped channel 187 by the closure top 18, the insert shank gripping portion 198 that is initially slidable along the shank surface 46 such that the stepped surfaces 40 digs or penetrates into the surface 198 and thus securely fixes the shank upper portion 8 to the insert 14, as seen in FIG. 27.

On either side of the arms 183′ and 183″ are flat surfaces 190′ and 190″. At the intersection of the surfaces 190′ and 190″ with the surfaces 183′ and 183″ are formed four corners or grooves 195. The corners 195 extend along a length of the insert 14 and are vertically or axially aligned and perpendicular to the flat surfaces 190′ and 190″ projecting outwardly. The corners 195 are sized and shaped to vertically slide, but snugly mate with the receiver guides 116, seen in FIG. 8. This allows the insert 14 to be directed to move vertically during loading into the receiver 10 and during certain positioning required during assembly and implantation of the bone screw assembly 1, but prevents the insert 14 from rotating about the axis C relative to the receiver 10. Referring to FIG. 20, the rear corners 195 are seen sliding vertically along the receiver interior 90 and guides 116, but are constrained from axial rotation by the abutment of the corners 195 with the guides 116.

The compression insert 14 also includes a first outer and upper cylindrical surface 202 adjacent to the outer surfaces 185 of the arms 183′ and 183″. The surface 202 may be continuous with and including the outer surfaces 185 of the insert arms 183′ and 183″, although this is not necessarily required. The insert 14 also includes a second outer lower and discontinuous cylindrical surface 204 adjacent to the bottom surface 194. A discontinuous annular transitional surface 205 extends between and connects the upper and lower cylindrical surfaces 202 and 204. The cylindrical surface 202 is sized and shaped to be received within the receiver interior surfaces 90 when loaded through the receiver bottom opening 136 as shown, for example, in FIG. 19. The lower outer discontinuous surface 204, on the other hand, has a neutral diameter that is larger than the diameters of the receiver bottom opening 136. Therefore, during assembly, the resilient insert fingers or panels 200 are pressed inwardly toward the receiver axis C to allow for insertion of the entire insert 14 into the receiver opening 136. As best shown in FIG. 19, the outer cylindrical surface 204 is sized and shaped so that once the insert 14 is in an operational position, and the panels 200 are frictionally mated about the shank upper portion 8, the second lower outer cylindrical surface 204 is in slidable engagement or slightly spaced from the receiver inner cylindrical wall 120.

It is foreseen that the insert 14 may not include arms 183′ and 183″. It is also foreseen that the arms 183′ and 183″ may be spaced from the closure top 18 in some embodiments and may be sized and shaped to contact the closure top 18 in other embodiments in order to provide locking of the polyaxial mechanism of the assembly with pivoting retainer 12, but without fixing of the rod or other longitudinal connecting member 21 with respect to the closure top 18.

Preferably, the receiver 10, the non-pivoting retainer 11, the wave spring 9, and the compression or pressure insert 14 are assembled at a factory setting that includes tooling for holding and alignment of the component pieces and pinching or compressing of the retainer 11, as well as, the wave spring 9. As described herein with respect to the assembly 1, similarly, the shank 4, which may be used in various embodiments with or without a pivoting retainer 12, may be assembled with the receiver 10, non-pivoting retainer 11 and compression or pressure insert 14 at the factory or it may be desirable to “pop” the shank 4 into the receiver subassembly at a later time, either before or after implantation of the shank 4 in the vertebra 13, scc FIGS. 29-31.

Pre-assembly of the receiver 10, non-pivoting retainer 11, wave spring 9, and compression insert 14 is shown in FIGS. 20-23. First, the compression insert 14 is uploaded into the receiver 10 through the lower opening 136 with the insert top surface 192 facing the receiver bottom surface 134. The receiver bottom opening diameter or width D3 or radius (not shown) is the same as or slightly smaller than the diameter or width D4 or radius (not shown) of the insert projection 184. Once the insert 14 enters the receiver cavity 41, the receiver guides 116 cooperate with the insert corners 195 to guide the insert 14 up and down in the receiver 10 while preventing rotation of the insert relative to the receiver 10. The insert 14 is slid upwardly toward the channel seat 88 with the panels 200 compressing inwardly as the insert 14 is moved upward until the receiver projecting structures 184 of the insert 14 snap into the insert attachment grooves 104 of the receiver interior 90 and the collet lower radial surface 204 is positioned within the fifth cylindrical surface area 120, as seen in FIG. 21. The resilient tabs 200 are released and the lower radial surface 204 of the tabs 200 remain spaced from the fifth cylindrical surface 120, so that the collet can expand to accept the shank head. At this point, the insert 14 is captured and frictionally held in place by the mating of the receiver attachment structures 184 within the insert attachment groove 104. This prevents the insert 14 from any unwanted downward movement or further upward movement towards the guide and advancement structure 92 of the receiver 10 and provides adequate clearance for the later step of pushing the bone screw head shank upper portion 8 through the non-pivoting retainer 11 and wave spring 9. Although the grooves 104 would prohibit the insert 14 from moving out the upper opening 86 of the receiver 10 and moving downward, engagement of the lower surfaces of the resilient tabs 200 with the cylindrical surface 122 in the receiver also prohibits downward movement of the insert 14 and keeps the insert 14 away from the lower opening 136 during assembly with the non-pivoting retainer 11, wave spring 9, and subsequent assembly with the shank 4. It is foreseen that a tool or tools (not shown) may be used to push up, pull or otherwise lift the insert into this position.

Referring now to FIGS. 10 and 22, the open wave spring 9 is prepared for insertion into the receiver 10 by squeezing or pressing or folding or compressing the wave spring end surfaces 174 and 175 toward one another. It is foreseen that the wave spring 9 may be inserted in various different methods. In one such method, the wave spring 9 may not allow for complete compression due to the size of gap X″, and the end surface 174 may need to overlap end surface 175 or vice versa prior to loading. Another method foreseen is uploading either end surface 174 or 175 into the receiver 10, and gradually loading a small portion of the wave spring 9 at a time around the expansion chamber from one end surface 174 to the other end surface 175 or vice versa. During this process the wave spring 9 is compressed diametrically at two points. The wave spring is typically moved upwardly into the receiver 10 and past the eighth cylindrical surface 127 and allowed to expand to a neutral uncompressed state within the expansion chamber 95 engaging seventh cylindrical surface 124, the stop surface 123 and the abutment surface 125. The peaks 167 engage the stop surface 123 and the valleys 168 engage the abutment surface 125.

Referring now to FIGS. 10 and 23, the resilient open non-pivoting retainer 11 is prepared for insertion into the receiver 10 by squeezing or pressing the retainer end surfaces 154 and 155 toward one another. The compressed retainer 11 is inserted into the lower opening 136 with the top surface 142 facing the receiver bottom surface 134. The retainer 11 is typically moved upwardly into the receiver 10 and is prevented or restricted from entering the expansion chamber 95 past the eighth cylindrical surface 127 and allowed to expand to a neutral uncompressed state within the eighth cylindrical surface 127 as shown in FIG. 24-26.

Also as shown in FIG. 23, at this time, the compression insert 14, the wave spring 9, and the non-pivoting retainer 11 are captured within the receiver 10. The receiver 10, compression insert 14, wave spring 9, and the non-pivoting retainer 11 combination are now pre-assembled and ready for shipment or assembly with the shank 4 either at the factory, by surgery staff prior to implantation, or directly after implanting the shank 4 by the surgeon.

In FIG. 24, the top surface 47 of the shank 4 is partially inserted into the expansion chamber 95, and the upper capture portion 8 of the shank 4 abuts against the non-pivoting retainer 11 held down in the locking seating chamber 99 of the receiver 10 by the wave spring 9. In FIG. 25, the non-pivoting retainer 11 has been lifted up into the expansion chamber 95 by the shank and has reached the maximum expansion about the shank capture portion 8, which is the center of the ball structure 44, at which point the outer surface 63 of the upper pivoting retainer 12 is positioned within the insert collect, so as to capture the shank upper capture portion 8 within the receiver 10.

In FIG. 26, the non-pivoting retainer ring 11 has captured the shank head after moving up into the expansion chamber 95 and then being forced downward by the wave spring 9 back down into the smaller diameter locking chamber 99 created by the eighth cylindrical surface 127 and the second stop surface 129, while the insert 14 remained in the fixed position aligned and prevented from rotating and moving vertically. The shank upper capture portion 8 at this point cannot be pulled out of the receiver 10.

In FIG. 27, at this time, the compression insert 14 is pressed downwardly by a tool, such as a screw driver (not shown), toward the shank upper portion 8 by the projection structure 186 out of the insert attachment groove or slot or aperture 104. This downward force is transferred to the top surface 162 of the wave spring 9, which in turn, applies downward pressure to the non-pivoting retainer 11 to maintain the non-pivoting retainer 11 in the lower second non-expanding locking seating chamber 99, further aiding in preventing the retainer from moving up or the shank upper capture portion 8 from coming out of the retainer lower opening 136. The insert 14 and the shank gripping portion 198 are in a fairly tight friction fit with the shank upper portion 8, and the stepped surface 40 on the top of the shank head firmly engages itself in the insert shank gripping portion 198, the stepped surface 40 still being pivotable with respect to the insert 14 with some force. Thus, a tight, non-floppy, substantially spherical ball and socket joint 44 is now created between the insert 14 and the shank upper portion 8 and the pivoting retainer 12 combination. At this time the non-pivoting retainer 11 has returned to a neutral position and is typically located within the eighth cylindrical wall or surface 127 of the locking chamber 99.

With reference to FIG. 28A, the second fixed position of the insert 14, once downward pressure is applied, creates an interference friction fit connection. The insert projection structure 184 is forced along the retainer surface, as shown in FIG. 20, and the upper portion of the third cylindrical surface 110 and a top surface 189 thereof, the insert projection structure engaging and abutting surface 189 in such a way as to maintain the insert in this position, but allowing the shank 4 to be further manipulated multiaxially. The friction fit between the compression insert 14 and the shank upper portion 8 and pivoting retainer 12 combination is not totally locked or fixed, but at the same time is not loose or floppy either, advantageously allowing the user to articulate the shank 4, in this case with a pivoting retainer 12, with respect to the receiver 10, but with some resistance, so that when the shank 4 is placed in a desired orientation with respect to the receiver 10, the assembly 1 remaining substantially frictionally set in such desired orientation unless purposefully manipulated into another position. For example, at this time, the receiver 10 may be articulated to a desired position with respect to the shank 4, for example, as shown in FIG. 31 or FIGS. 34 and 35, but prior to locking of such position that is shown in the latter drawings.

With reference to FIG. 28B, it is foreseen that a friction fit between the receiver 10 and the shank 4 may not be wanted by the surgeon. The projection structure 184 is positioned below the abutment surface 108, so as to allow the insert to move about the gap created by the third cylindrical surface 110, and thus creating a floppy fit between the receiver 10 and the shank 4. A floppy fit will allow the user to move the receiver 10 about the shank 4 freely, but will not stay in any one position chosen. To make the floppy fit stay, it will require the installation of a rod 21 and a closure 18.

With reference to FIG. 28C, it is foreseen that for an embodiment for the insert, the lower end of the collet portion does not extend around the hemisphere of the shank head with or without a pivoting retainer creating a gap Y.

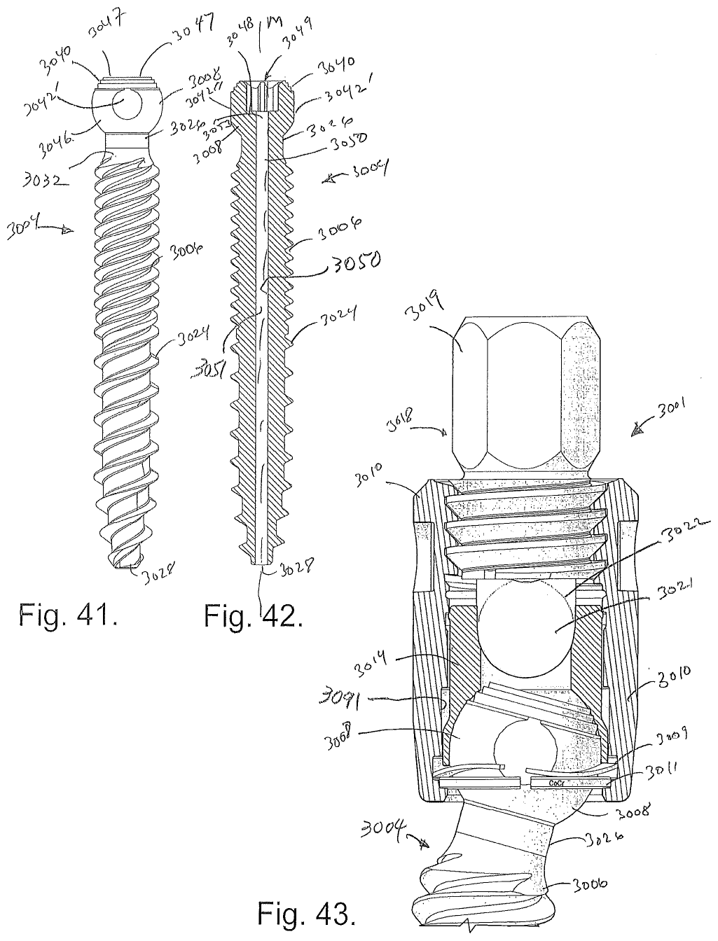

As illustrated in FIG. 29, the bone screw shank 4 (or an entire assembly 1 made up of the assembled shank 4 with or without, as shown in FIG. 41, the pivoting retainer 12, wave spring 9, receiver 10, non-pivoting retainer 11, and compression insert 14) is screwed into a bone, such as the vertebra 13, by rotation of the shank 4 using a suitable driving tool 29 or tool assembly (not shown) that operably drives and rotates the shank body 6 by engagement thereof at the internal drive 49. It is foreseen that the shank 4 and other bone screw assembly parts, the rod 21 (also having a central lumen in some embodiments) and the closure top 18 (also with a central bore drive) can be inserted in a percutaneous or minimally invasive surgical manner, utilizing guide wires (not shown) with or without minimally invasive guide tools.

Again, with respect to FIGS. 30 and 31, when the shank 4, and as shown, a pivoting retainer 12, in combination, are driven into the vertebra 13 without the remainder of the assembly 1, the shank 4 and pivoting retainer 12, when joined may either be driven to a desired final location or may be driven to a location slightly above or “proud” to provide for ease in assembly with the pre-assembled receiver 10, compression insert 14, wave spring 9, and non-pivoting retainer 11. With reference to FIG. 31, the pre-assembled receiver 10, insert 14, wave spring 9, and non-pivoting retainer 11 are placed above the shank upper portion 8 until the shank upper portion is received within the opening 136 of the receiver 10. As the shank upper capture portion 8 is moved into the interior 90 of the receiver base 80, the shank upper portion 8 presses the non-pivoting retainer 11 upwardly into the expansion chamber 95. As the shank upper capture portion 8 continues to move upwardly toward the channel 84, the retainer top surface 142 abuts against the valleys 167 of the wave spring 9, compressing the wave spring 9 and stopping upward movement of the non-pivoting retainer 11 and forcing expansion or outward movement of the retainer 11 towards the seventh cylindrical surface 124 defining the expansion chamber or groove 95 as the spherical outer surface 63 of the pivoting retainer 12 continues in an upward direction. The non-pivoting retainer 11 begins to contract about the spherical outer surface 63 as the widest region of the substantial sphere or ball 44 passes beyond the center of the retainer expansion chamber 95 (see FIG. 25). The retainer 11 is then pushed down into a final operative location within the seating locking chamber or groove 99, shown in FIG. 27 by either by the expansion of the wave spring 9 to the wave spring's original or nominal or neutral position, see FIG. 14A-B, or by the insert exerting pressure on the wave spring 9 to compress it against the non-pivoting retainer 11.

With reference to FIGS. 32-34, the rod 21 is eventually positioned in an open or percutaneous manner in cooperation with the at least two bone screw assemblies 1. The closure structure 18 is then inserted into and advanced between the arms 82 of each of the receivers 10.

The illustrated closure structure 18 is substantially cylindrical and includes an outer helically wound guide and advancement structure 78 in the form of a reverse angle thread form that operably joins with the guide and advancement structure 92 disposed on the arms 82 of the receiver 10, as seen in FIG. 10. The illustrated closure structure 18 also includes a top surface 74 with an internal drive (not shown) in the form of an aperture that may be star-shaped internal drive such as that sold under the trademark TORX, or may be, for example, a hex drive, or other non-round internal drives such as slotted, tri-wing, spanner, two or more apertures of various shapes, and the like. A driving tool (not shown) sized and shaped for engagement with the internal drive (not shown) is used for both rotatable engagement and, if needed, disengagement of the closure 18 from the receiver arms 82. The closure structure 18 includes a break-off head 19 designed to allow such a head to break from a base of the closure at a preselected torque, for example, 70 to 140 inch pounds. Such a closure structure would also include a base having an internal drive to be used for closure removal. A base or bottom surface 77 of the illustrated closure 18 is planar and may further include a point 75 for engagement and penetration into the surface 22 of the rod 21 in certain embodiments of the invention.

The closure structure 18 is rotated, using a hex breakoff head 19 engaged with tool (not shown) until a selected pressure is reached at which point the rod 21 engages the U-shaped channel 187 of the compression insert 14, further pressing the insert spherical surface 198 against the stepped surfaces 40 and the shank spherical surface 46, the edges of the stepped surfaces 40 penetrating into the spherical surface 198 or vice versa.

As the closure structure 18 rotates and moves downwardly into the respective receiver 10, the point 75 and bottom surface rim 77 engage and penetrate the rod surface 22, and the closure structure 18 presses downwardly against and biases the rod or connecting member 21 into engagement with the insert 14 that thereby urges the shank upper portion 8 toward the non-pivoting retainer 11 and into locking engagement therewith, with the non-pivoting retainer 11 frictionally abutting the surface 129 and expanding outwardly against the eighth cylindrical surface 127 of the locking chamber 99.

The closure top 18 may further include a cannulation through bore (not shown) extending along a central axis thereof and through the top and bottom surfaces thereof. Such a through bore provides a passage through the closure 18 interior for a length of wire (not shown) inserted therein to provide a guide for insertion of the closure top into the receiver arms 82, for example implantation of the assembly 1 during minimally invasive techniques. It is foreseen that any of a variety of different closure structures for use in conjunction with the present invention with suitable mating structure on the upstanding arms 82 can be utilized. For example, a closure having an outer ring (not shown) with a central screw (not shown) often referred to as a dual closure or dual innie so as to engage top surfaces (not shown) of upright arms (not shown) of an insert (not shown). Another example would be the present closure but without a break off head 19. Also multi-start threaded closures (not shown) are foreseen to be utilized in this invention.

With reference to FIGS. 32 and 33, different sizes of rods may be utilized with the same components of the bone screw assembly 1. In FIG. 32, a 4.5 mm rod 21 is utilized. In FIG. 33, a 4.75 mm rod 21′ is utilized. The only difference is the amount of rotation of the closure to fix the two embodiments of rods 21 and 21′. It is foreseen that inner cores (not shown) and sleeves (not shown) utilized in soft stabilization may also be used in this invention, such as those seen in U.S. patent application Ser. No. 14/731,064, the entirety of which is incorporated by reference herein.

With reference to FIG. 34-35, prior to locking the shank 4 with the closure 18, the shank 4 is multiaxially rotatable relative to the receiver 10 meaning that the angle of the shank 4 may be varied with respect to the receiver 10 and the axis A in two dimensions.

If removal of the rod 21 from any of the bone screw assemblies 1 is necessary, or if it is desired to release the rod 21 at a particular location, disassembly may be accomplished by using a driving tool (not shown) that mates with an internal drive (not shown) of the closure structure 18 to rotate and remove such closure structure from the cooperating receiver 10. Disassembly is then accomplished in reverse order to the procedure described previously herein for assembly. It is also foreseen that the implant may be a permanent fixture and not be dissembled, only removed as a whole assembled unit if necessary.

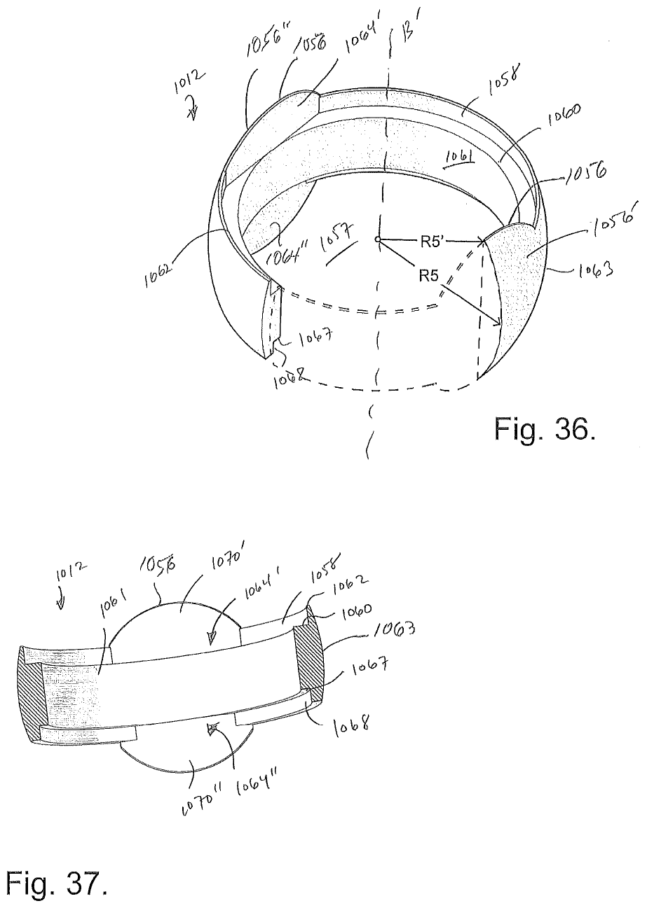

With particular reference to FIGS. 36-37, a modified pivoting retainer embodiment 1012 is shown. The pivoting retainer 1012 has a central axis B′ that is operationally similar to the axis B in pivoting retainer 12. The pivoting retainer 1012 is substantially similar to the pivoting retainer 12 as previously disclosed with the exception that a cylindrical surface 1061 is continuous about the interior surfaces that create the bore 1057 central to the discontinuous cylindrical surface 61 of the pivoting retainer 12, illustrated in FIG. 5.

It is foreseen that an outer spherical surface 1063 of the retainer 1012 may be smooth or alternatively include a roughened or textured surface or surface finish, or may be scored, knurled, grit blasted, or the like, for enhancing frictional engagement with the receiver 10. It is foreseen that the surfaces 1063 need not be spherical, but may be alternatively planar or faceted or include other surface geometries.

Referring now to FIG. 37, the illustrated pivoting retainer 1012 has a hollow through bore 1057 passing entirely through the pivoting retainer 1012. Surfaces that define the channel or bore 1057 include: an upper cylindrical surface 1058 adjacent to a retainer top surface 1062, a discontinuous shelf surface 1060 adjacent to the upper cylindrical surface 1058, the continuous cylindrical surface 1061 adjacent to the shelf surface 1060, a discontinuous sloped or ramped surface 1067 adjacent to the continuous cylindrical surface 1061, opposed inner planar surfaces 1064′ and 1064″ and communicating with the cylindrical surface 1058 and a third cylindrical surface 1068. The upper cylindrical surface 1058 is continuous about the bore 1057 of the pivoting retainer 1012, but for the inner planar surfaces 1064′ and 1064″. The inner planar surfaces 1064′ and 1064″ are the interior surfaces of circular enlargement 1056 of the pivoting retainer 1012, which have semi-spherically shaped surfaces 1056′ and 1056″ and include a circular portion 1070′ adjacent to the top surface 1062 communicating with the cylindrical surface 1058 and a circular portion 1070″ adjacent to a bottom surface or edge 1059 communicating with third cylindrical surface 1068. The inner planar surfaces 1064′ and 1064″ extend radially with axis B′ and are illustrated as being substantially tangential to the cylindrical surface 1061. In this embodiment, as the first and third discontinuous cylindrical surfaces 1058 and 1068 terminate at the inner planar surfaces 1064′ and 1064″, respectively. Unlike in the pivoting retainer 12 there is no gap or band between corresponding sets of surfaces, and the upper and lower shelf surfaces 1060 and 1067 terminate at a smaller angle than in the pivoting retainer 12 embodiment, such that cylindrical surface 1061 remains continuous. The inner surfaces 1064′ and 1064″ with circular portions 1070′ and 1070″ are sized and shaped to mate with and completely cover the opposed planar surfaces 42 (FIG. 2B) of the shank upper capture portion 8, as previously seen for the pivoting retainer 12. When the pivoting retainer 1012 is mated to shank upper capture portion 8, a substantially spherical ball component of the ball and socket structure 44 is created, shown in FIG. 26. Like the retainer 12 previously discussed, a radius R5 of the outer surface 1063 is the same as an upper radius R5′ of the spherical shape surfaces 1056′ and 1056″.

It is foreseen that further surfaces such as a lower shelf surface (not shown) and a fourth cylindrical surface (not shown) may be sized and shaped to further step down the spherical shape of the outer surface 1063 internally.

It is foreseen that the semi-spherically shaped surfaces 1056′ and 1056″ may include projections or notches as needed for tooling to resiliently hold the pivoting retainer 1012. It is foreseen that in other embodiments of the invention, fewer or greater number of planar or other surfaces with other geometries may extend between the top surface 1062 and the inner surfaces defining the bore 1057 of the pivoting retainer 1012.

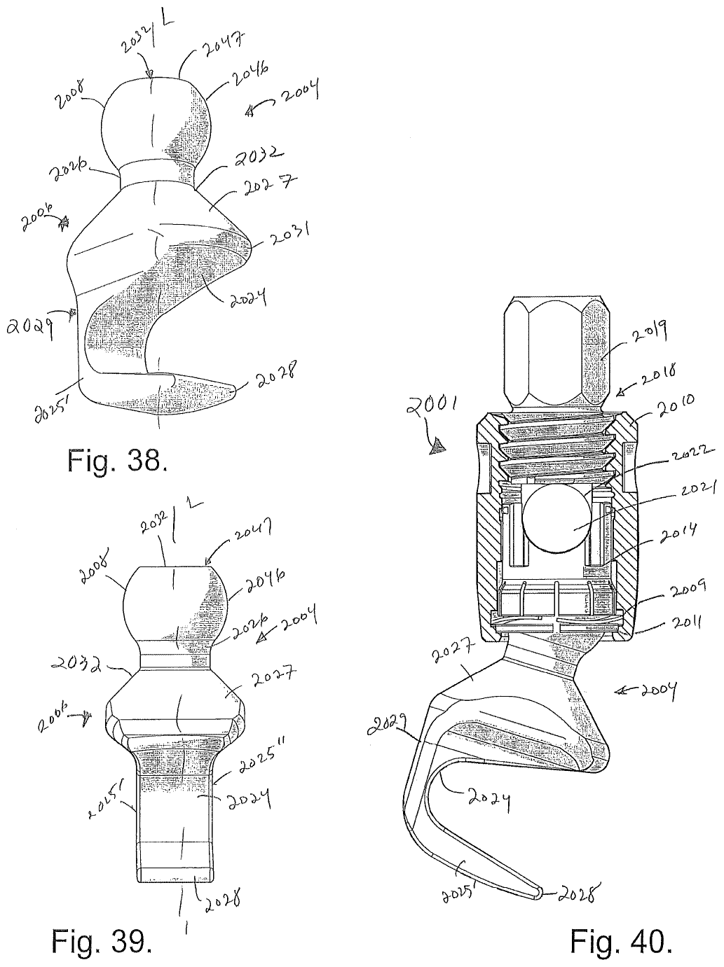

With reference to FIGS. 38-40, the reference number 2001 generally represents a polyaxial bone hook or anchor apparatus or assembly according to the present invention. The assembly 2001 includes a hook 2004, that further includes a body 2006 integral or monolithic with an upwardly extending upper portion 2008; a wave spring 2009, a receiver 2010; and a compression or pressure insert 2014. The receiver 2010, the wave spring 2009, the non-pivoting retainer 2011, and compression insert 2014 may be substantially identical to their counterparts of assembly 1, as discussed above and may be initially assembled and further assembled with the hook 2004, either prior or subsequent to implantation of the hook body 2006 on a vertebra (not shown) as discussed above. It is foreseen that the hook upper portion 2008 may be configured for use with a pivoting retainer (not shown) similar to the pivoting retainer 12.

Referring to FIGS. 38-40, the hook 2004 has a hook body 2006 and the upper capture portion 2008 and a neck 2026 that separates the hook body 2006 and the upper capture portion 2008. The hook 2004 has a central axis L. The neck 2026 extends axially along axis L and upwardly from the hook body 2006. The neck 2026 may be of the same or is typically of a slightly reduced radius as compared to an adjacent upper end or top 2032 of the body 2006 where the hook surfaces 2004 terminates. It is foreseen that the neck may be longer or shorter than what is shown, and that the hook may be replaced with an integral screw shank, like that shown in FIG. 1.

Further extending upwardly from the neck 2026 is the hook upper portion 2008 that provides a connective or capture apparatus adjacent from the upper end 2032 and thus at a distance from the vertebra (not shown) when the hook body 2006 is implanted on the vertebra.

The hook upper capture portion 2008 is configured for a pivotable connection between the hook 2004 and the receiver 2010 prior to fixing of the hook 2004 in a desired position with respect to the receiver 2010. The illustrated hook upper portion 2008 has a bulbous convex and at least partially spherical outer surface 2046 that extends outwardly and upwardly from the neck 2026 and terminates at a top surface 2047, the hook top surface 2047 may be substantially perpendicular to the axis L. The upper spherical surface 2046 shown in the present embodiment is substantially smooth. It is foreseen that the upper portion may further include a stepped or graduated upper surface portion (not shown) located adjacent to the top surface 2047 and sized and shaped for cooperation and ultimate frictional engagement with the compression insert 2014.

It is foreseen that a stepped or graduated upper surface portion (not shown) and also the rest of the spherical surface 2046 may additionally or alternatively include a roughened or textured surface or surface finish, or may be scored, knurled, grit blasted, or the like, for enhancing frictional engagement with the non-pivoting retainer 2011 and/or the compression insert 2014.

The hook body 2006 can have: a frusto-conical surface 2027 extending radially outward, wherein the frusto-conical surface 2027 being adjacent to the neck 2026, a partially cylindrical surface 2031 terminating at a back outer curved surface 2029, a curved inner surface 2024 extending from near the cylindrical surface 2031 to a tip 2028 of the body 2006, a pair of curved side surfaces 2025′ and 2025″ extending from the cylindrical surface 2031 to the tip 2028 following the curve of the curved inner surface 2024, and the outer curved surface 2029 continuing from the frusto-conical surface 2027 and following the curve of the inner curved surface 2024 to meet the pair of side curved surfaces 2025′ and 2025″ and the inner curved surface 2024 at the tip 2028.

During use, the body 2006 utilizes the inner curved surface 2034 for gripping a part of a vertebra (not shown) as the hook is implanted on the vertebra leading with the tip 2028 and curved around vertebra with the aid of a gripping or installation tool (not shown). It is foreseen that the inner curved surface 2024 may further include a stepped surface or teeth (not shown), so as to grip better to the vertebra (not shown). It is also foreseen that a tool engagement structure (not shown) on the hook may take a variety of tool-engaging forms and may include one or more apertures of various shapes, such as a pair of spaced apart apertures or horizontal grooves (not shown).

It is foreseen that the inner curved surface 2024, the pair of side curved surfaces 2025′ and 2025″, and the outer curved surface 2029 may have different lengths than illustrated. It is foreseen that the inner curved surface 2024, the pair of side curved surfaces 2025′ and 2025″, and the outer curved surface 2029 may have different widths, thereby creating a wider or thinner body 2006. It is foreseen that the inner curved surface 2024, the pair of side curved surfaces 2025′ and 2025″, and the outer curved surface 2029 may have different curvatures, such as, but not limited to a right angle, or left angle. The tip 2028 may be shortened or lengthened to stop at a different point, such that all of the inner curved surface 2024, the pair of side curved surfaces 2025′ and 2025″, and the outer curved surface 2029 may be affected.

FIG. 40 further shows a closure structure 2018 of the invention for capturing a longitudinal member 2021, for example, a rod 2021, which, in turn, engages the compression insert 2014 that presses the hook upper portion or capture portion 2008 into fixed frictional contact with the non-pivoting retainer 2011, so as to capture and fix the rod 2021 within the receiver 2010 and thus fix the rod 2021 relative to the vertebra (not shown). The illustrated rod 2021 is hard, stiff, non-elastic and cylindrical, having an outer cylindrical surface 2022. It is foreseen that in other embodiments, the rod 2021 may be elastic, deformable and/or of a different cross-sectional geometry. The receiver 2010 and the hook 2004 cooperate in such a manner that the receiver 2010 and the hook 2004 can be secured at any of a plurality of angles, articulations or rotational alignments relative to one another and within a selected range of angles from side to side and from front to rear, as seen in FIG. 40, to enable flexible or articulated engagement of the receiver 2010 with the hook 2004, which may then be locked or fixed relative to each other near the end of an implantation procedure.

To provide a biologically active interface with the bone, the hook body 2006 may be coated, perforated, made porous or otherwise treated. The treatment may include, but is not limited to: a plasma spray coating or other type of coating of a metal or, for example, a calcium phosphate; or a roughening, perforation or indentation in the hook surfaces, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄))₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

With reference to FIGS. 41-43, the reference number 3001 generally represents a polyaxial bone screw or anchor apparatus or assembly according to the present invention. The illustrated assembly 3001 includes a shank 3004, that further includes a body 3006 integral or monolithic with an upwardly extending upper capture portion 3008; a wave spring 3009, a receiver 3010; a non-pivoting retainer 3011, and a compression or pressure insert 3014. The receiver 3010, the wave spring 3009, the non-pivoting retainer 3011, and compression insert 3014 are substantially similar to their counterparts in assembly 1, as discussed above and may be initially assembled and further assembled with the shank 3004 either prior or subsequent to implantation of the shank body 3006 into a vertebra (not shown) as discussed above.

The shank 3004, best illustrated in FIGS. 41-42 is elongate, with the shank body 3006 having a helically wound bone implantable thread 3024 (single or multi-start thread form) extending from near a neck 3026 located adjacent to the upper portion or capture structure 3008, to a tip 3028 of the body 3006 and extending radially outwardly therefrom. The shank 3004 has a longitudinal axis, generally identified by the reference letter M, seen in FIG. 42.

Referring to FIGS. 41-42, the neck 3026 extends axially upward from the shank body 3006. The neck 3026 typically has a slightly reduced radius as compared to an adjacent upper end or top 3032 of the body 3006 where the thread 3024 terminates, although the neck 3026 may have the same radius as the upper end 3032. Further extending axially and outwardly from the neck 3026 is the shank upper portion 8 that provides a connective or capture apparatus disposed adjacent to the upper end 3032 and, thus, at a distance from the vertebra (not shown) when the body 3006 is implanted into the vertebra (not shown).

The shank upper capture portion 3008 is configured for a pivotable connection between the shank 3004 and the non-pivoting retainer 3011 and receiver 3010 prior to fixing of the receiver 3010 in a desired position with respect to the shank 3004. The shank upper portion 3008 has a bulbous, convex and partially spherical outer surface 3046 that extends outwardly and upwardly from the neck 3026 terminates at a top surface 3047.

The shank top surface 3047 is substantially perpendicular to the axis M. The upper spherical surface 3046 shown in the present embodiment is substantially smooth with the exception of a stepped or graduated upper surface portion 3040 located adjacent to the top surface 3047 and sized and shaped for cooperation and ultimate frictional engagement with the compression insert 3014. It is foreseen that the surface portion 3040 and also the rest of the spherical surface 3046 may additionally or alternatively include a roughened or textured surface or surface finish, or may be scored, knurled, grit blasted, or the like, for enhancing frictional engagement with the non-pivoting retainer 3011 and/or the compression insert 3014.

Referring to FIG. 42, formed in a shank upper portion 8 of the shank head 3004 are opposed and parallel flat planar surfaces 3042′ and 3042″ that extend downwardly in the direction of axis M, as is seen in U.S. Pat. App. U.S. patent application Ser. No. 14/061,393, the entirety of which is incorporated by reference. It is foreseen that the flat planar surfaces 3042′ and 3042″ may also include a lower key extension (not shown), as seen in U.S. patent application Ser. No. 13/573,516, which the entirety of is incorporated by reference herein. Once the shank head 3008 passes through the non-pivoting retainer 3011, the shank upper portion or head is captured within the receiver 3010.

A counter sunk, substantially planar base or seating surface 3048 partially defines an internal drive feature or imprint 3049. As best seen in FIG. 42, the illustrated internal drive feature 3049 is a closed aperture formed in the top surface 3047 and extending downwardly from the top surface 3047. The drive feature 3049 may have a hex shape designed to receive the hex tool (not shown) of an Allen wrench type, into the feature 3049 for rotating and driving the bone screw shank 3004. It is foreseen that such an internal tool engagement structure may take a variety of tool-engaging forms and may include one or more apertures of various shapes, such as a pair of spaced apart apertures or a multi-lobular or star-shaped aperture, such as those sold under the trademark TORX, or the like. It is foreseen that the drive structure 3049 may be made of a somewhat softer metal compared to that of the head. The seat or base 3048 of the drive feature 3049 is disposed perpendicular to the axis, with the drive feature 3049 otherwise being substantially coaxial with the axis M.

The shank 3004 shown in the drawings is cannulated, having a small central bore 3050 extending an entire length of the shank 3004 along the axis M. It is foreseen that the central bore 3050 may not extend in a parallel direction with M, or that the bore 3050 may not extend the entire length of the shank 3004. The bore 3050 is defined by an inner cylindrical surface 3051 of the shank 3004 and has a circular opening 3052 at the shank tip or end 3028 and an upper opening 3053 communicating with the external drive 3046 at the surface 3045. The illustrated bore 3050 is coaxial with the threaded body 3006 and the upper portion 3008. The bore 3050 provides a passage through the shank 3004 interior for a length of wire (not shown) inserted into the vertebra (prior to the insertion of the shank body 3006), the wire providing a guide for facilitated insertion of the shank body 3006 into the vertebra (not shown).

To provide a biologically active interface with the bone, the threaded shank body 3006 may be coated, perforated, made porous or otherwise treated. The treatment may include, but is not limited to: a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄))₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

With reference to FIGS. 44-47, the reference number 4001 generally represents a polyaxial bone screw or anchor apparatus or assembly according to the present invention. The assembly 4001 includes a shank 4004, that further includes a body 4006 integral with an upwardly extending upper portion 4008; a pivoting retainer 4012, a wave spring 4009, a receiver 4010; a non-pivoting retainer 4011, and a compression or pressure insert 4014. The shank 4004, receiver 4010, the wave spring 4009, the non-pivoting retainer 4011, and compression insert 4014 are substantially similar to their counterparts of the assembly 1, as discussed above and may be initially assembled and further assembled with the shank 4004 either prior or subsequent to implantation of the shank body 4006 into a vertebra (not shown), as discussed above. The receiver subassembly is a universal receiver subassembly among the envisioned and illustrated shanks, with or without pivoting retainers, but this principle is not meant to be limited specifically to the embodiments disclosed herein.

With particular reference to FIGS. 44-47, a pivoting retainer 4012 is shown in a third embodiment, wherein this retainer 4012 could be pre-loaded into a receiver 4010. The pivoting retainer 4012, again, operates to assist in capturing the shank upper portion 4008 within the receiver 4010. The pivoting retainer 4012 has a central axis P that is operationally the same as the axis A associated with the shank 4. The pivoting retainer 4012 is ring shaped having a central bore 4057 and made from a resilient material, such as a stainless steel or titanium alloy, so that a pivoting retainer body 4055 thereby may be expanded.

The pivoting retainer 4012 includes a substantially cylindrical continuous body 4055 except for a slot or slit 4054. The illustrated pivoting retainer 4012 has an outer radius R4 measured from the outer surface 4063 of the pivoting retainer 4012 to the axis P. The radius R4 is preferably equal to radii R1 and R2 previous disclosed with the shank 4 and the shank 4004. It is foreseen that the radius R4 may be slightly smaller or slightly larger than R1 and R2 or some combination thereof.

It is foreseen that the spherical surface 4063 may additionally or alternatively include a roughened or textured surface or surface finish, or may be scored, knurled, grit blasted, or the like, for enhancing frictional engagement with the receiver 4010. It is noted that the surfaces 4063 need not be spherical and may be planar or faceted or include other surface geometries.

In certain embodiments, the pivoting retainer body 4055 has a very narrow slit 4054 to be used for expansion purposes only when the shank upper capture portion 4008 is loaded into the retainer body 4055, or in some embodiments through a receiver lower opening 4136. It is foreseen that the pivoting retainer 4012 may need to be compressed to be fit into the cavity 4091 of the receiver 4010. The interior surfaces of the bore 4057 are sized and shaped such that the pivoting retainer body 4055 cannot compress further when mated with the shank upper capture portion 4008. The slit 4054 of the resilient pivoting retainer 4012 is defined by first and second end surfaces, 4065 and 4066, respectively, disposed in spaced relation to one another or they may also be touching when the retainer is in a neutral natural starting state or position. Both end surfaces 4065 and 4066 are disposed substantially perpendicular to a bottom surface 4059 and a top surface 4062. It is foreseen that the slit may be at an angle or curved. A width Z between the surfaces 4065 and 4066 is very narrow to provide stability to the pivoting retainer 4012 during operation.

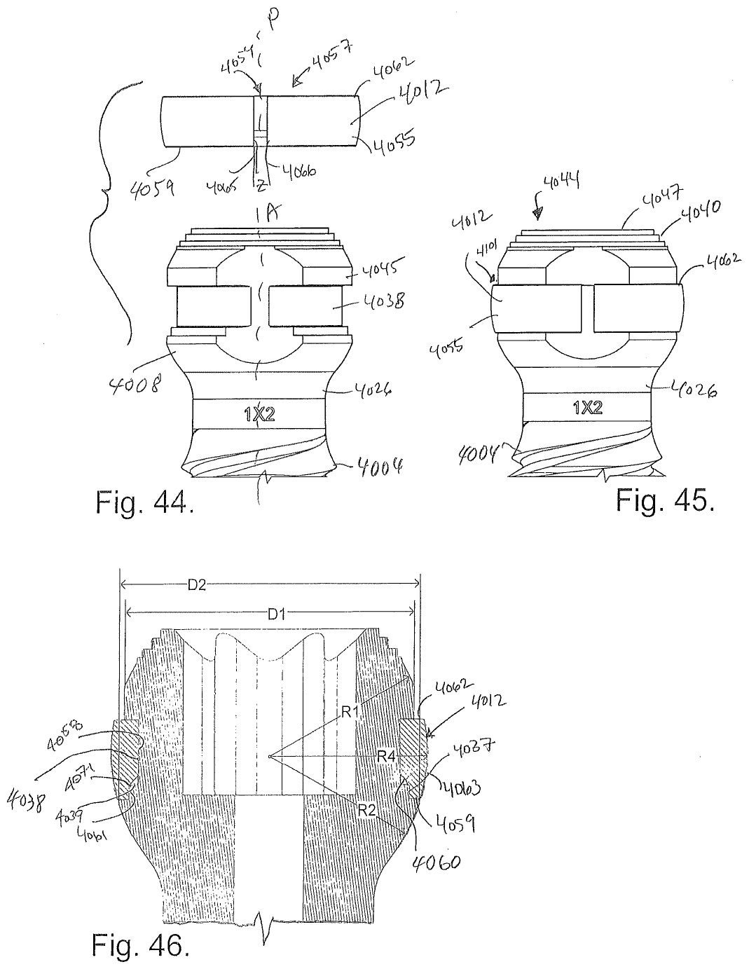

Referring now to FIG. 46, the pivoting retainer hollow through bore 4057 passes entirely through the pivoting retainer 4012. Interior surfaces that define the channel or bore 4057 include: an upper cylindrical surface 4058 adjacent to the retainer top surface 4062, a sloped or ramped shelf surface 4060 adjacent to the upper cylindrical surface 4058, a lower cylindrical surface 4061 adjacent to the sloped surface 4060 and the bottom surface 4059. As seen in FIGS. 45-46, the bore 4057 is sized and shaped to closely fit about and snap onto a shank interface surface 4038 during assembly. The upper cylindrical surface 4058 and the lower cylindrical surface 4061 are continuous about the bore 4057 of the pivoting retainer 4012. The sloped surface 4060 does not mate with the secondary ledge 4039 and secondary step surface 4037, but does engage an edge 4071 at the point where a secondary ledge 4039 and secondary step surface 4037 meet. When the pivoting retainer 4012 is mated to shank upper capture portion 4008, a partially spherical ball component of a ball and socket structure similar to the structure 44 shown in FIG. 26 is created. A gap 4101 exists where the top surface 4062 extends further than a cylindrical surface 4045 of the shank upper capture structure 4008. When mated with the shank upper capture portion 4008, the retainer top surface 4062 is situated below a top surface of the shank 4047. Again, the shank cylindrical surface 4045 is discontinuous and overlaps the top surface 4062 of the retainer 4012 on only opposite sides thereof.

It is foreseen that further interior surfaces such as a lower shelf surface (not shown) and a third cylindrical surface (not shown) may be sized and shaped to further step down the spherical shape of the outer surface 63 internally. It is foreseen that in other embodiments of the invention, fewer or greater number of planar or other surfaces with other geometries may extend between the top surface 4062 and the inner surfaces defining the bore 4057 of the pivoting retainer 4012, as will be discussed below.

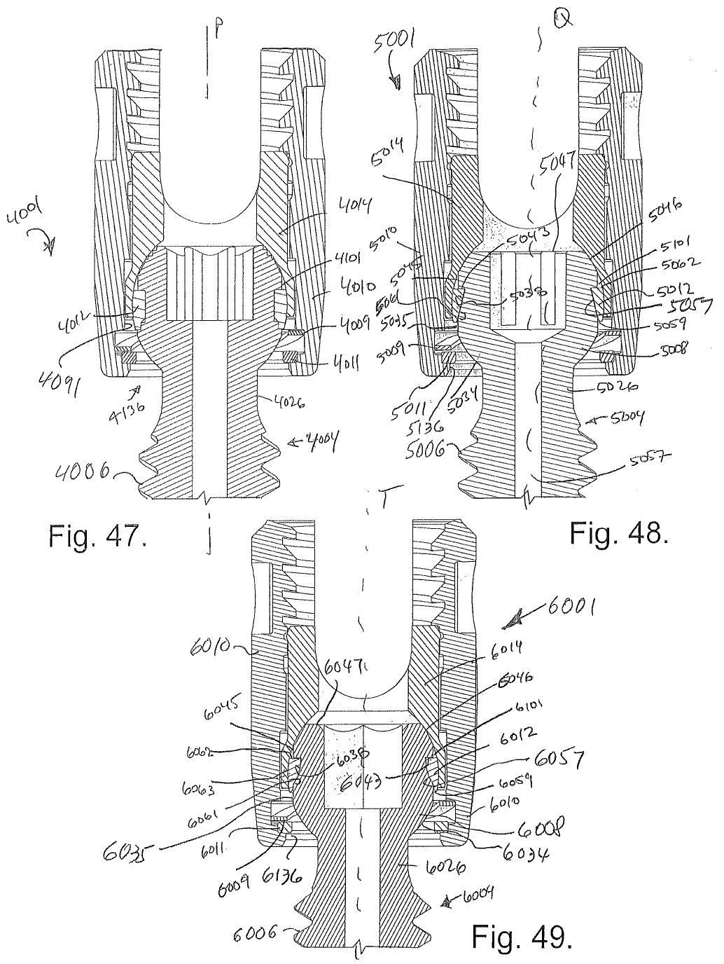

As best seen in FIG. 47, the pivoting retainer 4012 and shank upper capture portion 4008 in combination have been bottom loaded through the receiver lower opening 4136, such that the pivoting retainer 4012 is loaded in a neutral state and does not need to be further compressed to fit through the receiver lower opening 4136.

Referring now to FIG. 48, the reference number 5001 generally represents a polyaxial bone screw or anchor apparatus or assembly according to the present invention. The assembly 5001 includes a shank 5004, that further includes a body 5006 integral with an upwardly extending upper portion 5008; a pivoting retainer 5012, a wave spring 5009, a receiver 5010; a non-pivoting retainer 5011, and a compression or pressure insert 5014. The receiver 5010, the wave spring 5009, the non-pivoting retainer 5011, and compression insert 5014 are substantially similar to their counterparts in assembly 1, as discussed above and may be initially assembled and further assembled with the shank 5004 either prior or subsequent to implantation of the shank body 5006 into a vertebra (not shown), as discussed above.

In the illustrated embodiment of FIG. 48, the shank 5004 is substantially the same as the shank 4004 seen in FIGS. 44-47, with the exception of the interface between the pivoting retainer 5012 and the shank 5004 when they are mated and an upper spherical surface 5046, which is substantially spherical and does not further include stepped surfacing for interfacing with an insert 5014. The differences in the shank 5004 and pivoting retainer 5012 will be discussed below.

The shank upper capture portion 5008 is configured for a pivotable connection between the shank 5004 and the pivoting retainer 5012 and receiver 5010 prior to fixing of the shank 5004 in a desired position or axis or plane with respect to the receiver 5010. The shank upper portion 5008 has a bulbous convex and partially spherical outer lower surface 5034 that extends outwardly and upwardly from a neck 5026 terminates at a lower cylindrical surface 5035. The lower cylindrical surface 5035 is illustrated as parallel to axis Q. It is foreseen that the lower cylindrical surface 5035 may not be parallel with axis Q. The lower cylindrical surface 5035 terminates at a frusto-conical interface surface 5038. The frusto-conical interface surface 5038 is adjacent to an upper shelf 5043. The upper shelf surface 5043 is substantially perpendicular to the axis Q. The frusto-conical interface surface 5038 is defined as being wider near the lower cylindrical surface 5035 than at the upper shelf or ledge surface 5043. The spherical lower surface 5034 has an outer radius that is the same or substantially similar to an outer radius R1 of the shank 4004 and shank 4, as discussed above.

In this embodiment the frusto-conical interface surface 5038 and upper ledge 5043, which is, again, discontinuous, cooperate to capture and fix the resilient open pivoting retainer 5012 to the shank upper portion 5008, prohibiting compression of the pivoting retainer 5012 with respect to axis Q once the pivoting retainer 5012 is located underneath the ledge 5043. The top surface 5062 of the pivoting retainer 5012 interacts with the upper shelf surface 5043 and is beneath a top surface 5047 of the shank 5004. Extending upwardly from the upper ledge 5043 is a cylindrical surface 5045. The width or diameter or radius (not shown) of the cylindrical surface is the same as previously described D1 of shank 4, as seen in FIG. 2B.

Extending upwardly from the upper cylindrical surface 5045 is the upper partially spherical or domed surface 5046. The radius of the upper spherical surface 5046 may be the same as the radius R1 of the upper spherical surface 46 as previously discussed in FIG. 2B above. Located near or adjacent to the surface 5046 is the annular top surface 5047. It is foreseen that the upper spherical surface 5046 may be formed by stepped surfaces as was previously disclosed in shank 4 above.

The pivoting retainer 5012 is shown in a third embodiment with a first modification. The pivoting retainer 5012 operates to assist in capturing the shank upper portion 5008 within the receiver 5010, as previously discussed for the retainer 4012. The pivoting retainer 5012 may have an outer structure and slit (not shown) similar to the pivoting retainer 4012, but be adapted to mate with the frusto-conical interface surface 5038. The pivoting retainer 5012 has a central axis R that is operationally aligned with a longitudinal axis associated with the shank 5004 when mated. The illustrated pivoting retainer 5012 is ring shaped having a central bore 5057 and may be made from a resilient material, such as a stainless steel or titanium alloy, cobalt chrome, or the like, or a polymer, so that a pivoting retainer body 5055 may be expanded, either within or outside of the receiver 5010.

The pivoting retainer through bore 5057 passes entirely through the pivoting retainer 5012. The channel or bore 5057 is defined by the sloped or ramped or frusto-conical surface 5061. The interior frusto-conical surface 5061 is adjacent to a top surface 5062 and a bottom surface 5059 of the pivoting retainer 5012. As seen in FIG. 48, the bore 5057 is sized and shaped to closely fit about and snap onto the shank interface surface 5038 during assembly. The frusto-conical surface 5061 is continuous about the bore 5057 of the pivoting retainer 5012, which is similar to the first embodiment, assembly 1, which also included discontinuous surfaces to mate with the discontinuous surfaces of the interface surfaces of the shank 4. When the pivoting retainer 5012 is mated to shank upper capture portion 5008, a ball component of a ball and socket structure similar to the structure 44 (FIG. 26) is created. A gap 5101 exists where the top surface 5062 extends further than the cylindrical surface 5045 of the shank upper capture structure 5008.

It is foreseen that further interior surfaces such as a lower shelf surface (not shown) and a third cylindrical surface (not shown) may be sized and shaped to further step down the spherical shape of the outer surface 5063 internally.

As best seen in FIG. 48, the pivoting retainer 5012 and shank upper capture portion 5008 in combination have been bottom loaded through a receiver lower opening 5136, such that the pivoting retainer 5012 is loaded in a neutral State and does not need to be further compressed to fit through the receiver lower opening 5136.

Referring now to FIG. 49, the reference number 6001 generally represents a polyaxial bone screw or anchor apparatus or assembly according to the present invention. The assembly 6001 includes a shank 6004, that further includes a body 6006 integral with an upwardly extending upper portion 6008; a pivoting retainer 6012, a wave spring 6009, a receiver 6010; a non-pivoting retainer 6011, and a compression or pressure insert 6014. The receiver 6010, the wave spring 6009, the non-pivoting retainer 6011, and compression insert 6014 are substantially similar to their counterparts in the assembly 1, as discussed above and may be initially assembled and further assembled with the shank 6004 either prior or subsequent to implantation of the shank body 6006 into a vertebra (not shown), as discussed above.

In the illustrated embodiment of FIG. 49, the shank 6004 is substantially similar to shank 4004, and 5004 seen in FIGS. 44-48, with the exception of the interface between the pivoting retainer 6012 and the shank 6004 when they are mated. An upper spherical surface 6046 is substantially spherical and does not further include stepped surfacing for interfacing with an insert 6014. The differences in the shank 6004 and pivoting retainer 6012 will be discussed below.

The shank upper capture portion 6008 is configured for a pivotable connection between the shank 6004 and the pivoting retainer 6012 and receiver 6010 prior to fixing of the shank 6004 in a desired position or axis or plane with respect to the receiver 6010. The shank upper portion 6008 has a bulbous, convex and partially spherical lower outer surface 6034 that extends outwardly and upwardly from the neck 6026 terminates at a lower cylindrical surface 6035. The lower cylindrical surface 6035 is parallel to an axis T of assembly 6001. It is foreseen that the lower cylindrical surface does not have to be parallel with axis T. The lower cylindrical surface 6035 terminates at a curved interface surface 6038. The curved interface surface 6038 is adjacent to a downwardly facing upper shelf 6043. The upper shelf surface 6043 is also substantially perpendicular to the axis T. The curved interface surface 6038 is defined as being wider near the lower cylindrical surface 6035 than at the upper shelf or ledge surface 6043, much like the frusto-conical interface surface 6038 described above. The curved or curvate interface surface 6038 is not inclined at one specific angle like the frusto-conical interface surface 5038, but curves inwardly toward axis T from the lower cylindrical surface 6035 to the upper shelf surface 6043. It is foreseen that the curve of the curved surface may be outwardly sloped or curved inwardly at a steeper or more gradual incline or stepped or other geometric shapes. The spherical lower surface 6034 has an outer radius that is the same or substantially similar to an outer radius R1 of the shank 4004 and shank 4, as discussed above.

In this embodiment the curved interface surface 6038 and upper ledge 6043 cooperate to capture and fix the resilient open pivoting retainer 6012 to the shank upper portion 6008, prohibiting compression of the pivoting retainer 6012 with respect to axis T once the pivoting retainer 6012 is located underneath the ledge 6043. A top surface 6062 of the pivoting retainer 6012 interacts with the upper shelf surface 6043 and is located beneath a top surface 6047 of the shank 6004. Extending upwardly from the upper ledge 6043 is a cylindrical surface 6045. The width or diameter or radius (not shown) of the cylindrical surface may be the same as previously described D1 of shank 4, and is seen in FIG. 2B.

Extending upwardly from the upper cylindrical surface 6045 is an upper partially spherical or domed surface 6046. The radius of the upper spherical surface 6046 may be the same as the radius R1 of the upper spherical surface 46, as previously discussed in FIG. 2B above. Located near or adjacent to the surface 6046 is the annular top surface 6047. It is foreseen that the upper spherical surface may include stepped surfaces, as was previously disclosed in shank 4 above.

The pivoting retainer 6012 is shown in a third embodiment with a second modification. The pivoting retainer 6012 operates to assist in capturing the shank upper portion 6008 within the receiver 6010, as previously discussed for the retainer 4012 and 5012. The pivoting retainer 6012 may have the same outer structure and slit (not shown) as the pivoting retainer 4012, but must mate with the curved interface surface 6038. The pivoting retainer 6012 has a central axis (not shown) that is operationally aligned with a longitudinal axis associated with the shank 6004 when mated. The pivoting retainer 6012 is ring-shaped having a central bore 6057 and may be made from a resilient material, such as a stainless steel, cobalt chrome, or titanium alloy, or the like, or a polymer, or some combination, so that a pivoting retainer body 6055 may be expanded.

The pivoting retainer through bore 6057 passes entirely through the pivoting retainer 6012. The channel or bore 6057 is defined by an inwardly curved surface 6061. The interior curved surface 6061 is adjacent to the top surface 6062 and the bottom surface 6059 of the pivoting retainer 6012. As seen in FIG. 49, the bore 6057 is sized and shaped to closely fit about and snap onto the shank interface surface 6038 during assembly. The curved surface 6061 is continuous about the bore 6057 of the pivoting retainer 6012, which is, again, similar to the first embodiment assembly 1, which included discontinuous surfaces to mate with the discontinuous surfaces of the interface surfaces of the shank 4. When the pivoting retainer 6012 is mated to shank upper capture portion 6008, a partially spherical ball component of a ball and socket structure similar to the structure 44 seen in FIG. 26 is created. A gap 6101 exists where the top surface 6062 extends further than the cylindrical surface 6045 of the shank upper capture structure 5008.

It is foreseen that further interior surfaces such as a lower shelf surface (not shown) and a third cylindrical surface (not shown) may be sized and shaped to further step down the spherical shape of the outer surface 6063 internally. It is foreseen that the curve of the curved interior surface 6061 may be outwardly sloped or curved inwardly at a steeper or more gradual incline or stepped or other geometric shapes to mate with similar structure of the shank 6004.

As best seen in FIG. 49, the pivoting retainer 6012 and shank upper capture portion 6008 in combination have been bottom loaded through the receiver lower opening 6136, such that the pivoting retainer 6012 is loaded in a neutral state and does not need to be further compressed to fit through the receiver lower opening 6136.

It is to be understood that while certain forms of the present disclosure have been illustrated and described herein, it is not to be limited to the specific forms or arrangement of parts described and shown.

What is claimed is:

1. A pivotal bone anchor assembly intended for securing an elongate rod to a bone of a patient with tooling, the pivotal bone anchor assembly comprising:
a receiver comprising a vertical centerline axis, a lower portion defining a bottom opening communicating with a bottom surface of the lower portion, an upper portion, a central opening extending upwards from the bottom opening through the lower portion and the upper portion to a top surface thereof, internal surfaces of the central opening including a support surface adjacent to the bottom opening, a closure mating structure adjacent to a top of the receiver, opposed arcuate attachment grooves beneath the closure mating structure, and opposed arcuate recessed surfaces between the opposed arcuate attachment grooves and the support surface;
an insert disposable into the central opening of the receiver and comprising a top surface, an upper rod-receiving surface, a central through-bore, and opposite outwardly-facing side surfaces having arcuate projecting structures spaced above a bottom surface, the arcuate projecting structures being positionable in the opposed arcuate attachment grooves in a first position with upper and lower overlapping interference abutment surfaces therebetween configured to inhibit substantial axial movement in either direction within the central opening until forcibly engaged by the tooling; and
a bone attachment structure comprising a longitudinal axis, a capture head having a partial spherical surface defining a hemisphere plane at a greatest width perpendicular to the longitudinal axis, and an anchor portion opposite the capture head configured for attachment to the bone, the capture head being pivotally positionable in the lower portion of the receiver in an unlocked configuration,
wherein after the capture head of the bone anchor is positioned within the lower portion of the receiver, the insert is configured for forced downward displacement via direct engagement with the tooling to a second position defined by the arcuate projecting structures being at least partially adjacent to the opposed arcuate recessed surfaces and below the lower overlapping interference abutment surfaces to inhibit upward displacement of the insert back to the first position, and
wherein prior to locking the assembly with a closure top, the bone attachment structure is configured to remain pivotal with respect to the receiver in the unlocked configuration after the insert is forcibly downwardly displaced into the second position.

2. The pivotal bone anchor assembly of claim 1, wherein the receiver and the bone attachment structure have a non-floppy friction fit with respect to each other in the unlocked configuration after the insert is forcibly downwardly displaced into the second position.

3. The pivotal bone anchor assembly of claim 1, wherein the upper portion of the receiver includes a channel communicating with the central opening and configured to receive the elongate rod.

4. The pivotal bone anchor assembly of claim 1 and further comprising a retainer positionable in the lower portion of the receiver in engagement with the support surface and with the capture head of the bone attachment structure, the retainer being spaced apart from the insert after the insert is forcibly downwardly displaced into the second position.

5. The pivotal bone anchor assembly of claim 4, wherein the capture head of the bone attachment structure is uploaded through the bottom opening of the receiver and retained in the lower portion by the retainer.

6. The pivotal bone anchor assembly of claim 4, wherein the retainer is uploaded into the receiver through the bottom opening.

7. The pivotal bone anchor assembly of claim 1, wherein the insert is uploaded into the receiver through the bottom opening.

8. The pivotal bone anchor assembly of claim 7, wherein the internal surfaces of the central opening further comprise an insert guide and alignment structure preformed therein and vertically slidably mateable therewith upon positioning the insert into the receiver.

9. The pivotal bone anchor assembly of claim 1, wherein the insert further comprises an upper portion above a lower portion, the upper portion of the insert defining the upper rod-receiving surface and being devoid of a through-slot extending from one surface to an opposite surface of the insert on the upper portion.

10. The pivotal bone anchor assembly of claim 1, wherein the insert includes a downward-facing concave surface on a lower portion thereof configured for direct engagement with the capture head after the forced downward displacement of the insert within the central through-bore.

11. The pivotal bone anchor assembly of claim 1 and further comprising the elongate rod, wherein the closure top rotatably engageable with the closure mating structure to secure the elongate rod in the receiver with the pivotal bone anchor assembly in a locked configuration.

12. The pivotal bone anchor assembly of claim 11, wherein a release of the closure top with respect to the closure mating structure allows for further adjustment of the assembly in the unlocked configuration without utilization of tooling to engage the insert.
